(19) United States
(12) Patent Application Publication
(10) Pub. No.: US 2023/0210742 A1
(43) Pub. Date: Jul. 6, 2023

(54) CLEANSING COMPOSITIONS AND METHODS FOR USE

(71) Applicant: L'Oreal, Paris (FR)

(72) Inventors: Jun LIANG, Brooklyn, NY (US); Jianxin FENG, Clark, NJ (US); Siva MUTHUKRISHNAN, Bridgewater, NJ (US)

(21) Appl. No.: 18/091,017

(22) Filed: Dec. 29, 2022

Related U.S. Application Data
(60) Provisional application No. 63/295,694, filed on Dec. 31, 2021.

(30) Foreign Application Priority Data
Feb. 18, 2022 (FR) 2201436

(51) Int. Cl. A61K 8/46; A61K 8/60; A61K 8/44; A61Q 5/02

(52) U.S. Cl. CPC A61K 8/466; A61K 8/602; A61K 8/44; A61Q 5/02; A61K 2800/34; A61K 2800/596

(57) ABSTRACT

The disclosure relates to compositions for treating keratin materials. The compositions comprise (a) at least one amphoteric surfactant; (b) at least one anionic surfactant system comprising (i) at least one acyl taurate, (ii) at least one acyl isethionate, and (iii) at least one additional anionic surfactant; (c) at least one nonionic surfactant chosen from alkyl(poly)glycosides; (d) optionally, at least one cationic compound; and (e) sodium chloride, wherein the weight ratio of the (b) at least one anionic surfactant system to (e) sodium chloride ranges from about 5:1 to about 15:1. The disclosure also relates to methods of using the compositions.

### CROSS REFERENCE TO RELATED APPLICATIONS

This application claims priority to U.S. Provisional Application No. 63/295,694, filed Dec. 31, 2021, and FR 2201436 filed Feb. 18, 2022, both of which are incorporated herein in their entireties.

### TECHNICAL FIELD

The present disclosure relates to compositions for treating keratin materials, e.g. for cleansing keratin materials such as hair, and to methods for using the compositions.

### BACKGROUND

Conventional personal care cleansing compositions such as shampoo, body wash, facial cleanser, hand soap, etc., typically use sulfate-based surfactants such as sodium lauryl sulfate (SLS) or sodium laureth ether sulfate (SLES). These surfactants are commonly used because they have good foaming and cleansing properties, can be thickened easily, and are relatively inexpensive. However, there have been growing concerns in the marketplace over the negative effects of these or other sulfate-based surfactants, or sulfate-containing surfactants, on the skin and body. For example, sulfate-based surfactants have a tendency to dry out hair and skin, strip dye from color-treated hair, and break down proteins such as keratin, and may cause skin and eye irritation. In addition, SLES may contain dioxanes, byproducts generated in the manufacturing process, which are considered carcinogenic at high enough levels.

Silicones are commonly used in personal care products for their conditioning and cosmetic effects. For example, silicones provide a protective layer on the hair which allows the hair to be easily detangled and combed, and providing smoothness and glossiness. However, silicones can build up on hair layer-by-layer, which can weigh down the hair and make the hair greasy. Furthermore, silicones are not easily degraded, and accordingly their use in personal care products raises environmental concerns. Thus, it is desirable to maximize the positive properties provided by silicone and to minimize the negative environmental and build-up on hair. One way to achieve this is to limit or even eliminate silicones from cleansing compositions, whenever possible.

Furthermore, consumers desire natural compositions for personal care products such as compositions for cleansing hair and skin. There is an increased demand for sustainable, safe, and environmentally friendly “green” compositions that minimize the use of silicones, for example are free of or essentially free of silicones, as well as other synthetic chemical materials for cleansing and/or caring cleansing keratin materials, including hair and skin, and yet provide desirable overall good performance and high safety. However, such “green” compositions can be expensive to produce as their materials must be sourced from natural sources such as plants, as opposed to being high-volume, industrially-produced chemicals.

Therefore, cleansing compositions that are free of sulfate-based surfactants and essentially free of silicones or comprise limited quantities of silicones are becoming increasingly desirable to consumers. However, there are challenges in developing suitable formulations of cleansing products without the use of sulfate-based surfactants such as sulfate-based anionic surfactants (“sulfate-free”). For example, most existing sulfate-free hair cleansing products foam poorly, are opaque, and are not easily thickened. Traditional methods of increasing viscosity of these formulations are not effective with sulfate-free surfactants.

Thus, there is a need to overcome the above-described challenges for developing cleansing compositions, particularly cleansing compositions for the hair, that meet the consumers' increasing demands for natural and sulfate-free cleansing compositions that contain limited amounts of silicone, that also provide satisfactory cosmetic properties.

It has now surprisingly been found that, by using a unique combination of at least one amphoteric surfactant, an anionic surfactant system together with sodium chloride having a specific ratio range of anionic surfactant system to sodium chloride, an alkyl(poly)glucoside, and optionally a cationic compound, a cleansing composition, particularly a skin or hair cleansing composition that is free or substantially free of sulfate-based surfactants and minimizes the use of silicones, for example is free or essentially free of silicone compounds, can be prepared wherein the resulting cleansing composition requires lower amount of surfactant to produce excellent abundant and creamy foaming and cleansing properties, as well as good wet and dry cosmetic properties such as detangling and smoothness, and is environmentally friendly.

### SUMMARY

The present disclosure relates to compositions for treating keratin materials, particularly hair cleansing compositions that are free or substantially free of sulfates, and methods of using the compositions.

In various exemplary embodiments, there are provided compositions for cleansing keratin materials, e.g. skin, hair, etc., comprising at least one solvent and (a) at least one amphoteric surfactant; (b) an anionic surfactant system comprising: (i) at least one acyl taurate; (ii) at least one acyl isethionate; and (iii) at least one additional anionic surfactant, optionally chosen from alkoxylated monoacids, alkyl sulfosuccinates, alkyl ether sulfosuccinates, or mixtures thereof; (c) at least one nonionic surfactant chosen from alkyl(poly)glycosides; (d) optionally, at least one cationic compound; and (e) sodium chloride; wherein the weight ratio of the (b) anionic surfactant system to (e) sodium chloride ranges from about 5:1 to about 15:1; and wherein the composition is free or essentially free of sulfate-based surfactants. In some embodiments, the weight ratio of the (b) anionic surfactant system to (e) sodium chloride ranges from about 6:1 to about 14:1, from about 7:1 to about 13:1, from about 8:1 to about 12:1, or from about 8:1 to about 10:1.

In various embodiments, the total amount of sodium chloride ranges from about 0.01% to about 10%, such as from about 0.1% to about 5%, from about 0.5% to about 4%, or from about 0.75% to about 3% by weight, relative to the total weight of the composition.

In some embodiments, the compositions may further comprise (f) one or more silicones, and in other embodiments, the compositions may be free or essentially free of silicones. In exemplary embodiments where the compositions further comprise (f) at least one silicone, the total amount of silicone is typically less than about 2%, less than about 1.5%, less than about 1% or less than about 0.75%, or less than about 0.5% by weight, relative to the total weight of the composition. In various embodiments, the compositions comprise at least one thickening agent.

In various embodiments, the compositions comprise a total amount of cationic compounds ranging from about 0.01% to about 10%, from about 0.05% to about 5%, from about 0.075% to about 3%, or from about 0.1% to about 1% by weight, relative to the total weight of the composition.

In various embodiments, the compositions described herein comprise a total amount of amphoteric surfactants ranging from about 0.1% to about 15%, from about 1% to about 10%, from about 3% to about 8%, or from about 4% to about 7% by weight, relative to the total weight of the composition. In various embodiments, the (a) at least one amphoteric surfactant is chosen from (C₈-C₂₀) alkylbetaines, sulfobetaines, (C₈-C₂₀) alkylamido (C₆-C₈) alkylbetaines, (C₈-C₂₀) alkylamido (C₆-C₈) alkylsulfobetaines, salts thereof, or mixtures thereof, and in some embodiments the at least one amphoteric surfactant is chosen from coco betaine, cocoamidopropyl betaine, lauryl betaine, laurylhydroxy sulfobetaine, lauryldimethyl betaine, behenyl betaine, capryl/capramidopropyl betaine, stearyl betaine, salts thereof, or mixtures thereof.

In various embodiments, the compositions comprise a total amount of (b)(i) acyl taurates ranging from about 0.01% to about 8%, from about 0.05% to about 7%, from about 0.1% to about 5%, from about 0.5% to about 4%, or from about 0.75% to about 3% by weight, relative to the total weight of the composition. In various embodiments, the (b)(i) at least one acyl taurate is chosen from sodium methyl lauroyl taurate, sodium methyl cocoyl taurate, or mixtures thereof.

In various embodiments, the total amount of (b)(ii) acyl isethionates ranges from about 0.1% to about 15%, from about 0.5% to about 12%, from about 1% to about 10%, from about 2% to about 9%, or from about 3% to about 8% by weight, relative to the total weight of the composition.

In various embodiments, the total amount of (b)(iii) additional anionic surfactants chosen from alkoxylated monoacids, alkyl sulfosuccinates, alkyl ether sulfosuccinates, or mixtures thereof ranges from about 0.01% to about 10%, from about 0.05% to about 8%, from about 0.1% to about 5%, from about 0.25% to about 3%, or from about 0.5% to about 2.5% by weight, relative to the total weight of the composition. In certain embodiments, the (b)(iii) at least one additional anionic surfactant comprises at least one alkoxylated monoacid chosen from ceteareth-2 carboxylic acid, ceteareth-10 carboxylic acid, coceth-7 carboxylic acid, laureth-4 carboxylic acid, laureth-5 carboxylic acid, laureth-6 carboxylic acid, myreth-2 carboxylic acid, myreth-3 carboxylic acid, myreth-4 carboxylic acid, myreth-5 carboxylic acid, myreth-6 carboxylic acid, steareth-2 carboxylic acid, steareth-4 carboxylic acid, steareth-5 carboxylic acid, steareth-6 carboxylic acid, oleth-2 carboxylic acid, oleth-4 carboxylic acid, salts thereof, or mixtures thereof.

In various embodiments, the (c) at least one nonionic surfactant chosen from alkyl(poly)glycosides comprises at least one poly alkylglucoside chosen from decylglucoside, laurylglucoside, cocoglucoside, caprylylglucoside, caprylyl/capryl glucoside, or mixtures thereof. The total amount of nonionic surfactants chosen from alkyl(poly)glycosides may, in certain embodiments, range from about 0.1% to about 10%, from about 0.5% to about 8%, from about 1% to about 5%, or from about 1.5% to about 3% by weight, relative to the total weight of the composition.

In some embodiments, the compositions comprise at least one additional nonionic surfactant. In various embodiments, the additional nonionic surfactant(s), if present, may be chosen from alkyl and polyalkyl esters of poly(ethylene oxide), alkyl and polyalkyl ethers of poly(ethylene oxide), optionally polyoxyethylenated alkyl and polyalkyl esters of sorbitan, optionally polyoxyethylenated alkyl and polyalkyl ethers of sorbitan, alkyl and polyalkyl esters of sucrose, optionally polyoxyethylenated alkyl and polyalkyl esters of glycerol, optionally polyoxyethylenated alkyl and polyalkyl ethers of glycerol, or mixtures thereof.

In various exemplary embodiments, compositions for cleansing keratin materials comprise at least one solvent and (a) at least one amphoteric surfactant, present in an amount ranging from about 1% to about 15%, from about 3% to about 12%, or from about 5% to about 10%; (b) an anionic surfactant system comprising: (i) at least one acyl taurate, present in an amount ranging from about 0.1% to about 5%, from about 0.5% to about 4%, or from about 0.75% to about 3%; (ii) at least one acyl isethionate, present in an amount ranging from about 1% to about 10%, from about 2% to about 9%, or from about 3% to about 8%; and (iii) at least one additional anionic surfactant chosen from alkoxylated monoacids, alkyl sulfosuccinates, alkyl ether sulfosuccinates, or mixtures thereof, present in an amount ranging from about 0.1% to about 5%, from about 0.25% to about 3%, or from about 0.5% to about 2.5%; (c) at least one nonionic surfactant chosen from alkyl(poly)glycosides; (d) at least one cationic polymer; and (e) sodium chloride, wherein the weight ratio of the (b) anionic surfactant system to (e) sodium chloride ranges from about 6:1 to about 14:1, from about 7:1 to about 13:1, from about 8:1 to about 12:1, or from about 8:1 to about 10:1; wherein the amphoteric surfactant comprises at least one betaine; wherein the composition is free or essentially free of sulfate-based surfactants; and wherein all amounts are by weight, relative to the total weight of the composition.

In further exemplary embodiments, compositions for cleansing keratin materials comprise at least one solvent and (a) at least one betaine selected from coco betaine, cocamidoproyl betaine, salts thereof, or mixtures thereof, wherein the total amount of betaines ranges from about 1% to about 15%, from about 3% to about 12%, or from about 5% to about 10%; (b) an anionic surfactant system comprising: (i) sodium methyl cocoyl taurate, present in an amount ranging from about 0.1% to about 5%, from about 0.5% to about 4%, or from about 0.75% to about 3%; (ii) sodium cocoyl isethionate, present in an amount ranging from about 1% to about 10%, from about 2% to about 9%, or from about 3% to about 8%; and (iii) at least one alkoxylated monoacid, for example laureth-5 carboxylic acid, present in an amount ranging from about 0.1% to about 5%, from about 0.25% to about 3%, or from about 0.5% to about 2.5%; (c) at least one alkyl(poly)glycoside, present in an amount ranging from about 0.5% to about 8%, from about 1% to about 5%, or from about 1.5% to about 3%; (d) at least one cationic polymer; and (e) sodium chloride, wherein the weight ratio of the (b) anionic surfactant system to (e) sodium chloride ranges from about 8:1 to about 12:1, or from about 8:1 to about 10:1; wherein the composition is free or essentially free of sulfate-based surfactants; and wherein all amounts are by weight, relative to the total weight of the composition.

In various embodiments, the viscosity of the compositions as described herein ranges from about 800 cPs to about 20,000 cPs, such as from about 1000 cPs to about 18,000 cPs, from about 1500 cPs to about 15,000 cPs, or from about 2000 cPs to about 12,000 cPs.

The disclosure also relates to methods for cleansing keratin materials comprising applying a composition as described herein to the keratin materials, and subsequently rinsing the keratin materials. In preferred embodiments, the keratin material is hair.

In various embodiments, the weight ratio of anionic surfactant system to sodium chloride in the cleansing compositions of the invention ranges from about 5:1 to about 15:1, from about 5:1 to about 14:1, from about 5:1 to about 13:1, from about 5:1 to about 12:1, from about 5:1 to about 11:1, from about 5:1 to about 10:1, from about 5:1 to about 9:1, from about 6:1 to about 15:1, from about 6:1 to about 14:1, from about 6:1 to about 13:1, from about 6:1 to about 12:1, from about 6:1 to about 11:1, from about 6:1 to about 10:1, from about 6:1 to about 9:1, from about 7:1 to about 15:1, from about 7:1 to about 14:1, from about 7:1 to about 13:1, from about 7:1 to about 12:1, from about 7:1 to about 11:1, from about 7:1 to about 10:1, from about 7:1 to about 9:1, from about 8:1 to about 15:1, from about 8:1 to about 14:1, from about 8:1 to about 13:1, from about 8:1 to about 12:1, from about 8:1 to about 11:1, from about 8:1 to about 10:1, or from about 8:1 to about 9:1.

In some embodiments, the disclosure relates to compositions for cleansing keratin materials, e.g. skin, hair, etc., comprising at least one solvent and (a) at least one amphoteric surfactant, where the total amount of amphoteric surfactants ranges from about 0.1% to about 15%, such as from about 1% to about 10%, from about 3% to about 8%, or from about 4% to about 7% by weight, relative to the total weight of the composition, wherein the amphoteric surfactants are chosen from those of formulae (I)-(IV), for example chosen from (C₈-C₂₀) alkylbetaines, sulfobetaines, (C₈-C₂₀) alkylamido (C₆-C₈) alkylbetaines, (C₈-C₂₀) alkylamido (C₆-C₈) alkylsulfobetaines, salts thereof, or mixtures thereof, e.g. coco betaine, cocoamidopropyl betaine, lauryl betaine, laurylhydroxy sulfobetaine, lauryldimethyl betaine, behenyl betaine, capryl/capramidopropyl betaine, stearyl betaine, salts thereof, or mixtures thereof; (b) an anionic surfactant system comprising: (i) at least one acyl taurate, where the total amount of acyl taurates ranges from about 0.01% to about 8%, such as from about 0.05% to about 7%, from about 0.1% to about 5%, from about 0.5% to about 4%, or from about 0.75% to about 3% by weight, relative to the total weight of the composition, wherein the acyl taurates are chosen from those of formula (V), for example chosen from sodium methyl lauroyl taurate, sodium methyl cocoyl taurate, or mixtures thereof; (ii) at least one acyl isethionate, where the total amount of acyl isethionates ranges from about 0.1% to about 15%, such as from about 0.5% to about 12%, from about 1% to about 10%, from about 2% to about 9%, or from about 3% to about 8% by weight, relative to the total weight of the composition, wherein the acyl isethionates are chosen from those of formula (VII), for example chosen from sodium cocoyl isethionate, sodium cocoyl methyl isethionate, sodium lauroyl isethionate, sodium lauroyl methyl isethionate, sodium oleoyl isethionate, sodium oleoyl methyl isethionate, sodium stearoyl isethionate, sodium stearoyl methyl isethionate, sodium myristoyl isethionate, sodium myristoyl methyl isethionate, sodium palmitoyl isethionate, sodium palmitoyl methyl isethionate, a blend of stearic acid and sodium cocoyl isethionate, ammonium cocoyl isethionate, ammonium cocoyl methyl isethionate, or mixtures thereof; and (iii) at least one additional anionic surfactant chosen from alkoxylated monoacids, alkyl sulfosuccinates, alkyl ether sulfosuccinates, or mixtures thereof, for example chosen from alkoxylated monoacids of formula (VI), e.g. chosen from ceteareth-2 carboxylic acid, ceteareth-10 carboxylic acid, coceth-7 carboxylic acid, laureth-4 carboxylic acid, laureth-5 carboxylic acid, laureth-6 carboxylic acid, myreth-2 carboxylic acid, myreth-3 carboxylic acid, myreth-4 carboxylic acid, myreth-5 carboxylic acid, myreth-6 carboxylic acid, steareth-2 carboxylic acid, steareth-4 carboxylic acid, steareth-5 carboxylic acid, steareth-6 carboxylic acid, oleth-2 carboxylic acid, oleth-4 carboxylic acid, salts thereof, or mixtures thereof, where the total amount of additional anionic surfactants ranges from about 0.01% to about 10%, such as from about 0.05% to about 8%, from about 0.1% to about 5%, from about 0.25% to about 3%, or from about 0.5% to about 2.5% by weight, relative to the total weight of the composition; (c) at least one nonionic surfactant chosen from alkyl(poly)glycosides of formula (VIII), for example chosen from decylglucoside, laurylglucoside, cocoglucoside, caprylylglucoside, caprylyl/capryl glucoside, or mixtures thereof, wherein the total amount of nonionic surfactants of formula (VIII) ranges from about 0.1% to about 10%, such as from about 0.5% to about 8%, from about 1% to about 5%, or from about 1.5% to about 3% by weight, relative to the total weight of the composition; (d) at least one cationic compound, for example at least one cationic polymer, wherein the total amount of cationic compounds ranges from about 0.01% to about 10%, such as from about 0.05% to about 5%, from about 0.075% to about 3%, or from about 0.1% to about 1% by weight, relative to the total weight of the composition; (e) sodium chloride; (f) optionally at least one silicone compound, which, if present, may be chosen from polyorganosiloxanes, polyalkylsiloxanes, polyarylsiloxanes, polyalkarylsiloxanes, polyestersiloxanes, or mixtures thereof, for example chosen from dimethicone, cyclomethicone (cyclopentasiloxane), amodimethicone, cetearylamodimethicone, trimethyl silyl amodimethicone, phenyl trimethicone, trimethyl siloxy silicate, polymethylsilsesquioxane, or mixtures thereof, and may be present in an amount less than about 2%, such as less than about 1.5%, less than about 1% or less than about 0.75%, or less than about 0.5% by weight, relative to the total weight of the composition; (g) optionally at least one additional non-ionic surfactant, which, if present, may be chosen from alkyl and polyalkyl esters of poly(ethylene oxide), alkyl and polyalkyl ethers of poly(ethylene oxide), optionally polyoxyethylenated alkyl and polyalkyl esters of sorbitan, optionally polyoxyethylenated alkyl and polyalkyl ethers of sorbitan, alkyl and polyalkyl esters of sucrose, optionally polyoxyethylenated alkyl and polyalkyl esters of glycerol, and optionally polyoxyethylenated alkyl and polyalkyl ethers of glycerol, or mixtures thereof; and (h) optionally at least one thickener, wherein the weight ratio of the (b) anionic surfactant system to (e) sodium chloride ranges from about 5:1 to about 15:1, such as from about 6:1 to about 13:1, from about 7:1 to about 12:1, from about 8:1 to about 12:1, or from about 8:1 to about 10:1; and wherein the composition is free or essentially free of sulfate-based surfactants.

In further embodiments, the disclosure relates to compositions for cleansing keratin materials, e.g. skin, hair, etc., comprising at least one solvent and (a) at least one amphoteric surfactant chosen from betaines, for example chosen from coco betaine, cocoamidopropyl betaine, lauryl betaine, laurylhydroxy sulfobetaine, lauryldimethyl betaine, behenyl betaine, capryl/capramidopropyl betaine, stearyl betaine, salts thereof, or mixtures thereof, e.g. coco betaine and/or cocoamidopropyl betaine, where the total amount of amphoteric surfactants ranges from about 0.1% to about 15%, such as from about 1% to about 10%, from about 3% to about 8%, or from about 4% to about 7% by weight, relative to the total weight of the composition; (b) an anionic surfactant system comprising: (i) at least one acyl taurate chosen from sodium methyl lauroyl taurate and/or sodium methyl cocoyl taurate, where the total amount of acyl taurates ranges from about 0.01% to about 8%, such as from about 0.05% to about 7%, from about 0.1% to about 5%, from about 0.5% to about 4%, or from about 0.75% to about 3% by weight, relative to the total weight of the composition; (ii) at least one acyl isethionate chosen from sodium cocoyl isethionate, sodium cocoyl methyl isethionate, sodium lauroyl isethionate, sodium lauroyl methyl isethionate, sodium oleoyl isethionate, sodium oleoyl methyl isethionate, sodium stearoyl isethionate, sodium stearoyl methyl isethionate, sodium myristoyl isethionate, sodium myristoyl methyl isethionate, sodium palmitoyl isethionate, sodium palmitoyl methyl isethionate, a blend of stearic acid and sodium cocoyl isethionate, ammonium cocoyl isethionate, ammonium cocoyl methyl isethionate, or mixtures thereof, where the total amount of acyl isethionates ranges from about 0.1% to about 15%, such as from about 0.5% to about 12%, from about 1% to about 10%, from about 2% to about 9%, or from about 3% to about 8% by weight, relative to the total weight of the composition; and (iii) at least one additional anionic surfactant chosen from alkoxylated monoacids, alkyl sulfosuccinates, alkyl ether sulfosuccinates, or mixtures thereof, for example chosen from ceteareth-2 carboxylic acid, ceteareth-10 carboxylic acid, coceth-7 carboxylic acid, laureth-4 carboxylic acid, laureth-5 carboxylic acid, laureth-6 carboxylic acid, myreth-2 carboxylic acid, myreth-3 carboxylic acid, myreth-4 carboxylic acid, myreth-5 carboxylic acid, myreth-6 carboxylic acid, steareth-2 carboxylic acid, steareth-4 carboxylic acid, steareth-5 carboxylic acid, steareth-6 carboxylic acid, oleth-2 carboxylic acid, oleth-4 carboxylic acid, salts thereof, or mixtures thereof, e.g. comprising laureth-5 carboxylic acid, where the total amount of additional anionic surfactants ranges from about 0.01% to about 10%, such as from about 0.05% to about 8%, from about 0.1% to about 5%, from about 0.25% to about 3%, or from about 0.5% to about 2.5% by weight, relative to the total weight of the composition; (c) at least one nonionic surfactant chosen from decylglucoside, laurylglucoside, cocoglucoside, caprylylglucoside, caprylyl/capryl glucoside, or mixtures thereof, wherein the total amount of nonionic surfactants ranges from about 0.1% to about 10%, such as from about 0.5% to about 8%, from about 1% to about 5%, or from about 1.5% to about 3% by weight, relative to the total weight of the composition; (d) at least one cationic compound, for example at least one cationic polymer such as cationic guar gum derivatives, amidoamines, monoalkyl quaternary amines, dialkyl quaternary amines, polyquaternium compounds, or salts thereof, e.g. hydroxypropyl guar hydroxypropyltrimonium chloride, guar hydroxypropyltrimonium chloride, polyquaternium 4, polyquaternium 6, polyquaternium 7, polyquaternium 10, polyquaternium 11, polyquaternium 16, polyquaternium 22, polyquaternium 28, polyquaternium 32, polyquaternium-46, polyquaternium-51, polyquaternium-52, polyquaternium-53, polyquaternium-54, polyquaternium-55, polyquaternium-56, polyquaternium-57, polyquaternium-58, polyquaternium-59, polyquaternium-60, polyquaternium-63, polyquaternium-64, polyquaternium-65, polyquaternium-66, polyquaternium-67, polyquaternium-70, polyquaternium-73, polyquaternium-74, polyquaternium-75, polyquaternium-76, polyquaternium-77, polyquaternium-78, polyquaternium-79, polyquaternium-80, polyquaternium-81, polyquaternium-82, polyquaternium-84, polyquaternium-85, polyquaternium-86, polyquaternium-87, polyquaternium-90, polyquaternium-91, polyquaternium-92, or polyquaternium-94, or mixtures thereof, wherein the total amount of cationic compounds ranges from about 0.01% to about 10%, such as from about 0.05% to about 5%, from about 0.075% to about 3%, or from about 0.1% to about 1% by weight, relative to the total weight of the composition; (e) sodium chloride; (f) optionally at least one silicone compound, which, if present, may be chosen from polyorganosiloxanes, polyalkylsiloxanes, polyarylsiloxanes, polyalkarylsiloxanes, polyestersiloxanes, or mixtures thereof, for example chosen from dimethicone, cyclomethicone (cyclopentasiloxane), amodimethicone, cetearylamodimethicone, trimethyl silyl amodimethicone, phenyl trimethicone, trimethyl siloxy silicate, polymethylsilsesquioxane, or mixtures thereof, e.g. amodimethicone, and may be present in an amount less than about 2%, such as less than about 1.5%, less than about 1% or less than about 0.75%, or less than about 0.5%, for example is about 0.1%, about 0.2%, about 0.3%, or about 0.4% by weight, relative to the total weight of the composition; (g) optionally at least one additional non-ionic surfactant, which, if present, may be chosen from alkyl and polyalkyl esters of poly(ethylene oxide), alkyl and polyalkyl ethers of poly(ethylene oxide), optionally polyoxyethylenated alkyl and polyalkyl esters of sorbitan, optionally polyoxyethylenated alkyl and polyalkyl ethers of sorbitan, alkyl and polyalkyl esters of sucrose, optionally polyoxyethylenated alkyl and polyalkyl esters of glycerol, and optionally polyoxyethylenated alkyl and polyalkyl ethers of glycerol, or mixtures thereof; and (h) optionally at least one thickener, which, if present, may be chosen from starches and/or celluloses such as hydroxyethylcellulose, hydroxypropylmethylcellulose, and/or hydropropylcellulose, wherein the weight ratio of the (b) anionic surfactant system to (e) sodium chloride ranges from about 5:1 to about 15:1, such as from about 6:1 to about 13:1, from about 7:1 to about 12:1, from about 8:1 to about 12:1, or from about 8:1 to about 10:1; and wherein the composition is free or essentially free of sulfate-based surfactants.

In still further embodiments, the disclosure relates to compositions for cleansing keratin materials, e.g. skin, hair, etc., comprising at least one solvent and (a) at least one amphoteric surfactant chosen from coco betaine and/or cocoamidopropyl betaine, where the total amount of amphoteric surfactants ranges from about 0.1% to about 15%, such as from about 1% to about 10%, from about 3% to about 8%, or from about 4% to about 7% by weight, relative to the total weight of the composition; (b) an anionic surfactant system comprising: (i) at least one acyl taurate chosen from sodium methyl lauroyl taurate and/or sodium methyl cocoyl taurate, where the total amount of acyl taurates ranges from about 0.01% to about 8%, such as from about 0.05% to about 7%, from about 0.1% to about 5%, from about 0.5% to about 4%, or from about 0.75% to about 3% by weight, relative to the total weight of the composition; (ii) at least one acyl isethionate chosen from sodium cocoyl isethionate, where the total amount of acyl isethionates ranges from about 0.1% to about 15%, such as from about 0.5% to about 12%, from about 1% to about 10%, from about 2% to about 9%, or from about 3% to about 8% by weight, relative to the total weight of the composition; and (iii) at least one alkoxylated monoacids chosen from ceteareth-2 carboxylic acid, ceteareth-10 carboxylic acid, coceth-7 carboxylic acid, laureth-4 carboxylic acid, laureth-5 carboxylic acid, laureth-6 carboxylic acid, myreth-2 carboxylic acid, myreth-3 carboxylic acid, myreth-4 carboxylic acid, myreth-5 carboxylic acid, myreth-6 carboxylic acid, steareth-2 carboxylic acid, steareth-4 carboxylic acid, steareth-5 carboxylic acid, steareth-6 carboxylic acid, oleth-2 carboxylic acid, oleth-4 carboxylic acid, salts thereof, or mixtures thereof, where the total amount of additional anionic surfactants ranges from about 0.01% to about 10%, such as from about 0.05% to about 8%, from about 0.1% to about 5%, from about 0.25% to about 3%, or from about 0.5% to about 2.5% by weight, relative to the total weight of the composition; (c) at least one nonionic surfactant chosen from decylglucoside, laurylglucoside, cocoglucoside, caprylylglucoside, caprylyl/capryl glucoside, or mixtures thereof, wherein the total amount of nonionic surfactants ranges from about 0.1% to about 10%, such as from about 0.5% to about 8%, from about 1% to about 5%, or from about 1.5% to about 3% by weight, relative to the total weight of the composition; (d) at least one cationic compound chosen from hydroxypropyl guar hydroxypropyltrimonium chloride, guar hydroxypropyltrimonium chloride, polyquaternium 4, polyquaternium 6, polyquaternium 7, polyquaternium 10, polyquaternium 11, polyquaternium 16, polyquaternium 22, polyquaternium 28, polyquaternium 32, polyquaternium-46, polyquaternium-51, polyquaternium-52, polyquaternium-53, polyquaternium-54, polyquaternium-55, polyquaternium-56, polyquaternium-57, polyquaternium-58, polyquaternium-59, polyquaternium-60, polyquaternium-63, polyquaternium-64, polyquaternium-65, polyquaternium-66, polyquaternium-67, polyquaternium-70, polyquaternium-73, polyquaternium-74, polyquaternium-75, polyquaternium-76, polyquaternium-77, polyquaternium-78, polyquaternium-79, polyquaternium-80, polyquaternium-81, polyquaternium-82, polyquaternium-84, polyquaternium-85, polyquaternium-86, polyquaternium-87, polyquaternium-90, polyquaternium-91, polyquaternium-92, or polyquaternium-94, or mixtures thereof, for example comprising polyquaternium 7 and/or polyquaternium 10, wherein the total amount of cationic compounds ranges from about 0.01% to about 10%, such as from about 0.05% to about 5%, from about 0.075% to about 3%, or from about 0.1% to about 1% by weight, relative to the total weight of the composition; (e) sodium chloride; (f) at least one silicone compound chosen from polyorganosiloxanes, polyalkylsiloxanes, polyarylsiloxanes, polyalkarylsiloxanes, polyestersiloxanes, or mixtures thereof, for example chosen from dimethicone, cyclomethicone (cyclopentasiloxane), amodimethicone, cetearylamodimethicone, trimethyl silyl amodimethicone, phenyl trimethicone, trimethyl siloxy silicate, polymethylsilsesquioxane, or mixtures thereof, present in an amount less than about 2%, such as less than about 1.5%, less than about 1% or less than about 0.75%, or less than about 0.5%, for example is about 0.1%, about 0.2%, about 0.3%, or about 0.4% by weight, relative to the total weight of the composition; (g) optionally at least one additional non-ionic surfactant, which, if present, may be chosen from alkyl and polyalkyl esters of poly(ethylene oxide), alkyl and polyalkyl ethers of poly(ethylene oxide), optionally polyoxyethylenated alkyl and polyalkyl esters of sorbitan, optionally polyoxyethylenated alkyl and polyalkyl ethers of sorbitan, alkyl and polyalkyl esters of sucrose, optionally polyoxyethylenated alkyl and polyalkyl esters of glycerol, and optionally polyoxyethylenated alkyl and polyalkyl ethers of glycerol, or mixtures thereof; and (h) optionally at least one thickener, which, if present, may be chosen from starches and/or celluloses such as hydroxyethylcellulose, hydroxypropylmethylcellulose, and/or hydropropylcellulose, wherein the weight ratio of the (b) anionic surfactant system to (e) sodium chloride ranges from about 5:1 to about 15:1, such as from about 6:1 to about 13:1, from about 7:1 to about 12:1, from about 8:1 to about 12:1, or from about 8:1 to about 10:1; and wherein the composition is free or essentially free of sulfate-based surfactants.

### DESCRIPTION

The disclosure relates to compositions and methods for treating keratin materials. The compositions may be free or substantially free of sulfates, and are particularly useful as cleansing compositions, and in particular may be sulfate-free hair cleansing compositions that are free or substantially free of silicone. The applicants have surprisingly found that cleansing compositions according to the disclosure provide abundant and creamy foam, good conditioning, and cosmetic benefits for both wet and dry hair, even though the compositions are free or substantially free of sulfate-based surfactants and/or silicones.

I. Compositions

In exemplary and non-limiting embodiments, the disclosure relates to compositions, e.g. skin or hair cleansing compositions such as a shampoo, comprising (a) at least one amphoteric surfactant; (b) at least one anionic surfactant system comprising (i) at least one acyl taurate, (ii) at least one acyl isethionate, and (iii) at least one additional anionic surfactant; (c) at least one nonionic surfactant chosen from alkyl(poly)glycosides; (d) optionally, at least one cationic compound; and (e) sodium chloride, wherein the weight ratio of (b) anionic surfactant system to (e) sodium chloride ranges from about 5:1 to about 15:1.

For example, the weight ratio of (b) anionic surfactant system to (e) sodium chloride may range from about 5:1 to about 14:1, from about 5:1 to about 13:1, from about 5:1 to about 12:1, from about 5:1 to about 11:1, from about 5:1 to about 10:1, from about 5:1 to about 9:1, from about 6:1 to about 15:1, from about 6:1 to about 14:1, from about 6:1 to about 13:1, from about 6:1 to about 12:1, from about 6:1 to about 11:1, from about 6:1 to about 10:1, from about 6:1 to about 9:1, from about 7:1 to about 15:1, from about 7:1 to about 14:1, from about 7:1 to about 13:1, from about 7:1 to about 12:1, from about 7:1 to about 11:1, from about 7:1 to about 10:1, from about 7:1 to about 9:1, or from about 8:1 to about 9:1.

In exemplary embodiments, the compositions may further comprise (f) at least one silicone, and/or (g) at least one additional non-ionic surfactant, and/or (h) at least one thickener.

Compositions according to the disclosure are typically free or essentially of sulfate-based surfactants. In some embodiments, the compositions include silicones, and in other embodiments the compositions are free or essentially free of silicones. In further embodiments, compositions according to the disclosure are free or essentially free of both sulfate-based surfactants and silicones. In still further embodiments, the compositions are free or essentially free of sulfate-based surfactants, and include at least one silicone wherein the total amount of silicone compounds is not greater than about 2% by weight, relative to the total weight of the composition.

### Amphoteric Surfactants

Compositions according to the disclosure comprise at least one amphoteric surfactant chosen from betaines, salts thereof, or mixtures thereof.

In various embodiments, non-limiting examples of betaines or salts thereof the at least one amphoteric surfactant may comprise alkyl betaines, amido betaines, or mixtures thereof. In various embodiments, the compositions comprise at least one compound chosen from (C₈-C₂₀) alkylbetaines, sulfobetaines, (C₈-C₂₀) alkylamido (C₆-C₈) alkylbetaines, (C₈-C₂₀) alkylamido (C₆-C₈) alkylsulfobetaines, salts thereof, or mixtures thereof.

In some embodiments, exemplary useful betaines include, but are not limited to, those of the following formulae (I)-(IV):

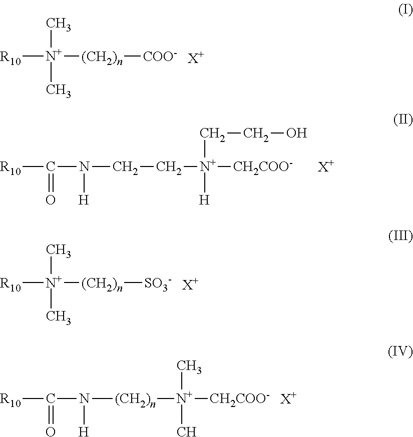

wherein:

R₁₀ is an alkyl group having from 8 to 18 carbon atoms;

n is an integer ranging from 1 to 3; and

X⁺ is a cationic counterion.

Particularly useful betaines include, for example, cocobetaine, cocamidopropyl betaine, cetyl betaine, lauryl betaine, laurylhydroxy sulfobetaine, lauryldimethyl betaine, cocamidopropyl hydroxysultaine, behenyl betaine, capryl/capramidopropyl betaine, lauryl hydroxysultaine, stearyl betaine, salts thereof, or mixtures thereof. In some embodiments, betaines include cocobetaine, cocamidopropyl betaine, salts thereof, or mixtures thereof.

In various embodiments, the total amount of betaines or salts thereof in the composition may vary, but typically ranges from about 0.1% to about 15% by weight, including all subranges therebetween, such as from about 1% to about 10%, from about 3% to about 8%, or from about 4% to about 7% by weight, relative to the total weight of the composition. In various embodiments, the total amount of betaines or salts thereof ranges from about 0.1% to about 14%, from about 0.1% to about 13%, from about 0.1% to about 12%, from about 0.1% to about 11%, from about 0.1% to about 10%, from about 0.1% to about 9%, from about 0.1% to about 8%, from about 0.1% to about 7%, from about 1% to about 15%, from about 1% to about 14%, from about 1% to about 13%, from about 1% to about 12%, from about 1% to about 11%, from about 1% to about 10%, from about 1% to about 9%, from about 1% to about 8%, from about 1% to about 7%, from about 2% to about 15%, from about 2% to about 14%, from about 2% to about 13%, from about 2% to about 12%, from about 2% to about 11%, from about 2% to about 10%, from about 2% to about 9%, from about 2% to about 8%, from about 2% to about 7%, from about 3% to about 15%, from about 3% to about 14%, from about 3% to about 13%, from about 3% to about 12%, from about 3% to about 11%, from about 3% to about 10%, from about 3% to about 9%, from about 3% to about 7%, from about 4% to about 15%, from about 4% to about 14%, from about 4% to about 13%, from about 4% to about 12%, from about 4% to about 11%, from about 4% to about 10%, from about 4% to about 9%, from about 4% to about 8%, from about 5% to about 15%, from about 5% to about 14%, from about 5% to about 13%, from about 5% to about 12%, from about 5% to about 11%, from about 5% to about 10%, from about 5% to about 9%, from about 5% to about 8%, from about 5% to about 7%, or from about 5% to about 6% by weight, relative to the total weight of the composition.

The betaines may, in some embodiments, be the predominant surfactant component in the composition.

### Anionic Surfactant System

Compositions according to the disclosure comprise an anionic surfactant system. The anionic surfactant system comprises (i) at least one acyl taurate or a salt thereof, (ii) at least one acyl isethionate or a salt thereof, and (iii) at least one additional anionic surfactant, which may, in some embodiments, be chosen from alkoxylated monoacids, alkyl sulfosuccinates, alkyl ether sulfosuccinates, salts thereof, or mixtures thereof.

### Acyl Taurates

Anionic surfactant systems according to the disclosure comprise at least one acyl taurate. Non-limiting examples of acyl taurates include those of formula (V), salts thereof, or mixtures thereof:

RCO—NR¹CHR²CHR³SO₃X⁺ (V)

wherein R, R¹, R², and R³ are each independently selected from H or an alkyl chain having from 1-24 carbon atoms, such as from 6-20 carbon atoms, or from 8-16 carbon atoms, said chain being saturated or unsaturated, linear or branched, substituted or unsubstituted, and X⁺ is a cationic counterion.

In various embodiments, RCO— in formula (V) represents the coconut acid moiety.

For example, acyl taurates may be chosen from compounds of formula (VI), salts thereof, or mixtures thereof:

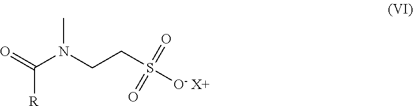

wherein:

R is chosen from hydrogen or a saturated or unsaturated, linear or branched alkyl chain having from 1 to 24 carbon atoms, preferably from 6 to 20 carbon atoms, more preferably from 8 to 16 carbon atoms; and

X⁺ is a cationic counterion, preferably chosen from sodium.

Non-limiting examples of acyl taurate salts that may be chosen include sodium methyl cocoyl taurate, sodium methyl lauroyl taurate, and mixtures thereof.

In various embodiments, the compositions comprise a total amount of acyl taurates ranging from about 0.01% to about 8%, such as from about 0.05% to about 7%, from about 0.1% to about 5%, from about 0.5% to about 4%, or from about 0.75% to about 3% by weight, relative to the total weight of the composition. In particular embodiments, the compositions comprise from about 0.01% to about 7%, from about 0.01% to about 6%, from about 0.01% to about 5%, from about 0.01% to about 4%, from about 0.01% to about 3%, from about 0.01% to about 2%, from about 0.05% to about 6%, from about 0.05% to about 5%, from about 0.05% to about 4%, from about 0.05% to about 3%, from about 0.05% to about 2%, from about 0.05% to about 1%, from about 0.1% to about 8%, from about 0.1% to about 7%, from about 0.1% to about 6%, from about 0.1% to about 5%, from about 0.1% to about 4%, from about 0.1% to about 3%, or from about 0.1% to about 2% by weight, relative to the total weight of the composition.

### Acyl Isethionates

Anionic surfactant systems according to the disclosure comprise at least one acyl isethionate. Non-limiting examples of acyl isethionates and their salts include those of formula (VII), salts thereof, or mixtures thereof:

RCOOCHR¹CHR²X⁻M⁺ (VII)

wherein:

R, R¹, and R² are independently chosen from hydrogen or an alkyl chain having from 1-24 carbon atoms, said chain being saturated or unsaturated, linear or branched;

X is SO₃⁻; and

M⁺ is any suitable cationic counterion.

Although the cation may be chosen from any suitable cation including, for example, alkali metal ion such as sodium or potassium, ammonium ions, or alkanolammonium ions such as monoethanolammonium or triethanolammonium ions, sodium is a preferred cation in at least some embodiments.

In various embodiments, RCO— in formula (VII) represents the coconut acid moiety.

According to various embodiments, the at least one isethionate surfactant may be chosen from acyl isethionates of the following formula (VIII), salts thereof, or mixtures thereof:

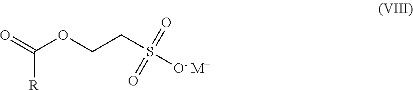

wherein:

R is chosen from hydrogen or a saturated or unsaturated, linear or branched alkyl chain having from 1 to 24 carbon atoms, preferably from 6 to 20 carbon atoms, more preferably from 8 to 16 carbon atoms; and

M⁺ is a cationic counterion.

Although the cation may be chosen from any suitable cation including, for example, alkali metal ion such as sodium or potassium, ammonium ions, or alkanolammonium ions such as monoethanolammonium or triethanolammonium ions, sodium is a preferred cation in at least some embodiments.

By way of non-limiting example, suitable acyl isethionate surfactants may include the reaction product of fatty acids esterified with isethionic acid and neutralized with sodium hydroxide. For example, acyl isethionates surfactants may be prepared by the reaction of an isethionate salt such as metal or ammonium isethionate and an a saturated or unsaturated, straight or branched, alkyl or alkenyl chain fatty acid having from 6 to 30 carbon atoms, preferably from 8 to 22 carbon atoms, more preferably from 6 to 18 carbon atoms. Optionally, a mixture of aliphatic fatty acids may be used for the preparation of commercial fatty acyl isethionates surfactants. Suitable fatty acids for isethionate surfactants can be derived from coconut oil or palm kernel oil, for instance.

Non-limiting examples of acyl isethionate surfactants that may be used include sodium cocoyl isethionate, sodium cocoyl methyl isethionate, sodium lauroyl isethionate, sodium lauroyl methyl isethionate, sodium oleoyl isethionate, sodium oleoyl methyl isethionate, sodium stearoyl isethionate, sodium stearoyl methyl isethionate, sodium myristoyl isethionate, sodium myristoyl methyl isethionate, sodium palmitoyl isethionate, sodium palmitoyl methyl isethionate, a blend of stearic acid and sodium cocoyl isethionate, ammonium cocoyl isethionate, ammonium cocoyl methyl isethionate, or mixtures thereof. In some embodiments, the compositions comprise sodium cocoyl isethionate.

The total amount of isethionate surfactants in the cleansing compositions of the invention may range up to about 15%, such as from about 0.1% to about 15%, from about 0.5% to about 12%, from about 1% to about 10%, from about 2% to about 9%, or from about 3% to about 8% by weight, relative to the total weight of the composition. For example, the total amount of isethionate surfactants may range from about 0.1% to about 14%, from about 0.1% to about 13%, from about 0.1% to about 12%, from about 0.1% to about 11%, from about 0.1% to about 10%, from about 0.1% to about 9%, from about 0.1% to about 8%, from about 0.1% to about 7%, from about 0.1% to about 6%, from about 0.5% to about 14%, from about 0.5% to about 13%, from about 0.5% to about 12%, from about 0.5% to about 11%, from about 0.5% to about 10%, from about 0.5% to about 9%, from about 0.5% to about 8%, from about 0.5% to about 7%, from about 0.5% to about 6%, from about 1% to about 15%, from about 1% to about 14%, from about 1% to about 13%, from about 1% to about 12%, from about 1% to about 11%, from about 1% to about 10%, from about 1% to about 9%, from about 1% to about 8%, from about 1% to about 7%, from about 1% to about 6%, from about 2% to about 15%, from about 2% to about 14%, from about 2% to about 13%, from about 2% to about 12%, from about 2% to about 11%, from about 2% to about 10%, from about 2% to about 9%, from about 2% to about 8%, from about 2% to about 7%, from about 2% to about 6%, from about 3% to about 15%, from about 3% to about 14%, from about 3% to about 13%, from about 3% to about 12%, from about 3% to about 11%, from about 3% to about 10%, from about 3% to about 9%, from about 3% to about 8%, from about 3% to about 7%, from about 3% to about 6%, from about 4% to about 15%, from about 4% to about 14%, from about 4% to about 13%, from about 4% to about 12%, from about 4% to about 11%, from about 4% to about 10%, from about 4% to about 9%, from about 4% to about 8%, from about 4% to about 7%, from about 4% to about 6%, from about 5% to about 15%, from about 5% to about 14%, from about 5% to about 13%, from about 5% to about 12%, from about 5% to about 11%, from about 5% to about 10%, from about 5% to about 9%, from about 5% to about 8%, from about 5% to about 7%, from about 5% to about 6%, from about 5.5% to about 15%, from about 5.5% to about 14%, about 5.5% to about 13%, about 5.5% to about 12%, about 5.5% to about 11%, from about 5.5% to about 10%, from about 5.5% to about 9%, from about 5.5% to about 8%, from about 5.5% to about 7%, about 6% to about 15%, about 6% to about 14%, about 6% to about 13%, about 6% to about 12%, about 6% to about 11%, about 6% to about 10%, from about 6% to about 9%, from about 6% to about 8%, or from about 6% to about 7% by weight, relative to the total weight of the composition, including ranges and sub-ranges there between.

### Additional Anionic Surfactant(s)

The anionic surfactant systems in compositions according to the disclosure comprise at least one additional anionic surfactant in addition to the aforementioned acyl taurates and acyl isethionates. In various, non-limiting embodiments, the at least one additional anionic surfactant is chosen from alkoxylated monoacids, alkyl sulfosuccinates, alkyl ether sulfosuccinates, salts thereof, or mixtures thereof. In some embodiments, the at least one additional anionic surfactant comprises, consists essentially or, or consists of one or more alkoxylated monoacids.

Alkoxylated Monoacids

Non-limiting examples of alkoxylated monoacids include compounds corresponding to formula (IX), salts thereof, or mixtures thereof:

R—O[CH₂O]ᵤ[(CH₂)ₓCH(R′)(CH₂)_{y}(CH₂)_{z}O]ᵥ[CH₂CH₂O]_{w}CH₂COOH (IX)

wherein:

R is a hydrocarbon radical containing from about 6 to about 40 carbon atoms;

R′ represents hydrogen or alkyl;

u, v, and w, which may be identical or different, independently represent numbers from 0 to 60;

x, y, and z, which may be identical or different, independently represent numbers from 0 to 13; and

the sum of x+y+z>0.

Compounds corresponding to formula (IX) can be obtained by alkoxylation of alcohols R—OH with ethylene oxide as the sole alkoxide, or with several alkoxides and subsequent oxidation. The numbers u, v, and w each represent the degree of alkoxylation. Whereas, on a molecular level, the numbers u, v, and w and the total degree of alkoxylation can only be integers, including zero, on a macroscopic level they are mean values in the form of broken numbers.

In formula (IX), R is linear or branched, acyclic or cyclic, saturated or unsaturated, aliphatic or aromatic, substituted or unsubstituted. For example, R may be a linear or branched, acyclic C6-C40 alkyl or alkenyl group or a C1-C40 alkyl phenyl group, more typically a C6-C22 alkyl or alkenyl group, or a C4-C18 alkyl phenyl group, for example a C12-C18 alkyl group or alkenyl group or a C6-C16 alkyl phenyl group. Further, u, v, w, independently of one another, may be chosen from a number ranging from 0 to 20, such as a number ranging from 3 to 17, or a number ranging from 5 to 15. In some embodiments, u and v are 0 and w is 5. Further still, x, y, z, independently of one another, may be chosen from a number ranging from 0 to 13, such as a number ranging from 1 to 10, or a number ranging from 2 to 8.

Suitable alkoxylated monoacids include, but are not limited to: Laureth-3 Carboxylic Acid, Laureth-4 Carboxylic Acid, Laureth-5 Carboxylic Acid, Laureth-6 Carboxylic Acid, Laureth-8 Carboxylic Acid, Laureth-10 Carboxylic Acid, Laureth-11 Carboxylic Acid, Laureth-12 Carboxylic Acid, Laureth-13 Carboxylic Acid, Laureth-14 Carboxylic Acid, Laureth-17 Carboxylic Acid, PPG-6-Laureth-6 Carboxylic Acid, Butoxynol-5 Carboxylic Acid, Butoxynol-19 Carboxylic Acid, Capryleth-4 Carboxylic Acid, Capryleth-6 Carboxylic Acid, Capryleth-9 Carboxylic Acid, Ceteareth-25 Carboxylic Acid, Coceth-7 Carboxylic Acid, C9-11 Pareth-6 Carboxylic Acid, C11-15 Pareth-7 Carboxylic Acid, C12-13 Pareth-5 Carboxylic Acid, C12-13 Pareth-8 Carboxylic Acid, C12-13 Pareth-12 Carboxylic Acid, C12-15 Pareth-7 Carboxylic Acid, C12-15 Pareth-8 Carboxylic Acid, C14-15 Pareth-8 Carboxylic Acid, Deceth-7 Carboxylic Acid, PPG-8-Steareth-7 Carboxylic Acid, Myreth-3 Carboxylic Acid, Myreth-5 Carboxylic Acid, Nonoxynol-5 Carboxylic Acid, Nonoxynol-8 Carboxylic Acid, Nonoxynol-10 Carboxylic Acid, Octeth-3 Carboxylic Acid, Octoxynol-20 Carboxylic Acid, Oleth-3 Carboxylic Acid, Oleth-6 Carboxylic Acid, Oleth-10 Carboxylic Acid, PPG-3-Deceth-2 Carboxylic Acid, Capryleth-2 Carboxylic Acid, Ceteth-13 Carboxylic Acid, Deceth-2 Carboxylic Acid, Hexeth-4 Carboxylic Acid, Isosteareth-6 Carboxylic Acid, Isosteareth-11 Carboxylic Acid, Trudeceth-3 Carboxylic Acid, Trideceth-6 Carboxylic Acid, Trideceth-8 Carboxylic Acid, Trideceth-12 Carboxylic Acid, Trideceth-3 Carboxylic Acid, Trideceth-4 Carboxylic Acid, Trideceth-7 Carboxylic Acid, Trideceth-15 Carboxylic Acid, Trideceth-19 Carboxylic Acid, Undeceth-5 Carboxylic Acid, or mixtures thereof. In some cases, preferred ethoxylated acids include Oleth-10 Carboxylic Acid, Laureth-5 Carboxylic Acid, Laureth-11 Carboxylic Acid, or mixtures thereof.

In various embodiments, compositions according to the disclosure comprise a total amount of alkoxylated monoacids ranging from about 0.01% to about 10%, such as from about 0.05% to about 8%, from about 0.1% to about 5%, from about 0.25% to about 3%, or from about 0.5% to about 2.5% by weight, relative to the total weight of the composition. In various embodiments, the compositions comprise a total amount of alkoxylated monoacids ranging from about 0.01% to about 9%, from about 0.01% to about 8%, from about 0.01% to about 7%, from about 0.01% to about 6%, from about 0.01% to about 5%, from about 0.01% to about 4%, from about 0.01% to about 3%, from about 0.01% to about 2%, from about 0.01% to about 1%, from about 0.05% to about 7%, from about 0.05% to about 6%, from about 0.05% to about 5%, from about 0.05% to about 4%, from about 0.05% to about 3%, from about 0.05% to about 2%, from about 0.05% to about 1%, from about 0.1% to about 7%, from about 0.1% to about 6%, from about 0.1% to about 5%, from about 0.1% to about 4%, from about 0.1% to about 3%, from about 0.1% to about 2%, from about 0.1% to about 1%, from about 0.5% to about 7%, from about 0.5% to about 6%, from about 0.5% to about 5%, from about 0.5% to about 4%, from about 0.5% to about 3%, from about 0.5% to about 2.5%, from about 0.5% to about 2%, from about 0.5% to about 1%, from about 0.25% to about 3%, or from about 0.25% to about 2.5% by weight, relative to the total weight of the composition.

Alkyl Sulfosuccinates

Compositions according to the disclosure may comprise at least one alkyl sulfosuccinate as an additional anionic surfactant. In some embodiments, however, the compositions are free or essentially free of alkyl sulfosuccinates.

Non-limiting examples of useful alkyl sulfosuccinates and their salts include those of formula (X), salts thereof, or mixtures thereof:

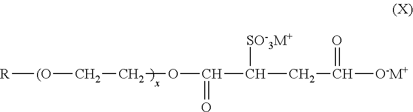

wherein:

R is a straight or branched chain alkyl or alkenyl group having 10 to 22 carbon atoms, preferably 10 to 20 carbon atoms;

x is a number that represents the average degree of ethoxylation, and can range from 0 to about 5, preferably from 0 to about 4, and most preferably from about 2 to about 3.5; and

M⁺, which may be the same or different, is chosen from cationic counterions.

In some embodiments, cations are alkali metal ions such as sodium or potassium, ammonium ions, or alkanolammonium ions such as monoethanolammonium or triethanolammonium ions.

Non-limiting examples of alkyl sulfosuccinates salts include disodium oleamido MIPA sulfosuccinate, disodium oleamido MEA sulfosuccinate, disodium lauryl sulfosuccinate, disodium laureth sulfosuccinate, diammonium lauryl sulfosuccinate, diammonium laureth sulfosuccinate, dioctyl sodium sulfosuccinate, disodium oleamide MEA sulfosuccinate, sodium dialkyl sulfosuccinate, or mixtures thereof.

In various embodiments, the total amount of each anionic surfactant in the anionic surfactant system may vary, but the anionic surfactant system is typically present in the composition in a total amount ranging from about 0.01% to about 20% by weight, including all subranges therebetween, such as from about 1% to about 18%, from about 2% to about 15%, from about 3% to about 12%, or from about 5% to about 10% by weight, relative to the total weight of the composition. In some embodiments, the total amount of anionic surfactants in the compositions may range from about 7% to about 10% by weight, relative to the total weight of the composition.

### Alkyl(poly)glucoside(s)

Compositions according to the disclosure comprise at least one nonionic surfactant chosen from alkyl(poly)glucosides. As used herein, the term “alkyl(poly)glucoside” refers to alkyl and polyalkyl glucosides, and polyglucosides.

Alkyl or polyalkyl glucosides or polyglucosides that may be used include, for example, those containing an alkyl group comprising from 6 to 30 carbon atoms, preferably from 6 to 18 or even from 8 to 16 carbon atoms, and containing a glucoside group which preferably comprises from 1 to 5, for example 1, 2, or 3 glucoside units. In certain embodiments, the compositions comprise at least one alkyl polyglucoside having the following formula (XI):

R₁O—(R₂O)ₜ—(G)v (XI)

wherein:

R₁ represents a linear or branched, saturated or unsaturated alkyl group, containing from 6 to 30, for example from 8 to 24, preferably from 8 to 18 carbon atoms, or an alkyl phenyl group in which the linear or branched alkyl radical contains from 8 to 24 carbon atoms;

R₂ represents an alkylene group containing from about 2 to 4 carbon atoms;

G represents a saccharide unit containing 5 or 6 carbon atoms, preferably glucose, fructose, or galactose;

t is a number ranging from 0 to 10, preferably from 0 to 4, more preferably from 0 to 3; and

v is a number ranging from 1 to 15.

Nonlimiting examples of useful alkyl(poly)glucosides include lauryl glucoside, octyl glucoside, decyl glucoside, coco glucoside, caprylyl/capryl glucoside, and sodium lauryl glucose carboxylate. In certain embodiments, the at least one alkyl poly glucoside compound is selected from the group consisting of lauryl glucoside, decyl glucoside, coco glucoside, and mixtures thereof. In some embodiments, the compositions comprise decyl glucoside.

In various embodiments, the total amount of alkyl(poly)glycosides ranges from about 0.1% to about 10%, such as from about 0.5% to about 8%, from about 1% to about 5%, or from about 1.5% to about 3% by weight, relative to the total weight of the composition.

### Cationic Compound(s)

Optionally, compositions according to the disclosure comprise at least one cationic compound, although in some embodiments the compositions are free or essentially free of cationic compounds. The cationic compounds may be chosen from cationic polymers, and cationic conditioning compounds other than cationic polymers. The term “cationic polymer” means any polymer comprising at least one cationic group and/or at least one group that may be ionized into a cationic group. In various embodiments, the cationic compound(s) may be chosen from cationic guar gum derivatives, amidoamines, monoalkyl quaternary amines, dialkyl quaternary amines, polyquaternium compounds, or salts thereof.

Exemplary and non-limiting cationic guar gum derivatives include hydroxypropyl guar hydroxypropyltrimonium chloride, guar hydroxypropyltrimonium chloride, or a mixture thereof.

Examples of amidoamines that are useful in the compositions of the instant disclosure include, but are not limited to the following: oleamidopropyl dimethylamine, stearamidopropyl dimethylamine, isostearamidopropyl dimethylamine, stearamidoethyl dimethylamine, lauramidopropyl dimethylamine, myristamidopropyl dimethylamine, behenamidopropyl dimethylamine, dilinoleamidopropyl dimethylamine, palmitamidopropyl dimethylamine, ricinoleamindopropyl dimethylamine, soyamidopropyl dimethylamine, wheat germamidopropyl dimethylamine, sunflowerseedamidopropyl dimethylamine, almondamidopropyl dimethylamine, avocadoamidopropyl dimethylamine, babassuamidopropyl dimethylamine, cocamidopropyl dimethylamine, minkamidopropyl dimethylamine, oatamidopropyl dimethylamine, sesamidopropyl dimethylamine, tallamidopropyl dimethylamine, brassicamidopropyl dimethylamine, olivamidopropyl dimethylamine, palmitamidopropyl dimethylamine, stearamidoethyldiethylamine, and mixtures thereof.

In further embodiments, cationic compound may be chosen from polyquaterium-10 (also called quaternized polyhydroxyethyl cellulose), cetrimonium chloride, behentrimonium chloride, behentrimonium methosulfate, steartrimonium chloride, stearalkonium chloride, dicetyldimonium chloride, hydroxypropyltrimonium chloride, cocotrimonium methosulfate, olealkonium chloride, steartrimonium chloride, babassuamidopropalkonium chloride, brassicamidopropyl dimethylamine, Quaternium-91, Salcare/PQ-37, Quaternium-22, Quaternium-87, lauryl betaine, Polyacrylate-1 Crosspolymer, steardimonium hydroxypropyl hydrolyzed wheat protein, behenamidopropyl PG-dimonium chloride, lauryldimonium hydroxypropyl hydrolyzed soy protein, Quaterium-8, or mixtures thereof.

In various embodiments, cationic compounds that may be used include, but are not limited to: polyquaterniums such as polyquaternium 4, polyquaternium 6, polyquaternium 7, polyquaternium 10, polyquaternium 11, polyquaternium 16, polyquaternium 22, polyquaternium 28, polyquaternium 32, polyquaternium-46, polyquaternium-51, polyquaternium-52, polyquaternium-53, polyquaternium-54, polyquaternium-55, polyquaternium-56, polyquaternium-57, polyquaternium-58, polyquaternium-59, polyquaternium-60, polyquaternium-63, polyquaternium-64, polyquaternium-65, polyquaternium-66, polyquaternium-67, polyquaternium-70, polyquaternium-73, polyquaternium-74, polyquaternium-75, polyquaternium-76, polyquaternium-77, polyquaternium-78, polyquaternium-79, polyquaternium-80, polyquaternium-81, polyquaternium-82, polyquaternium-84, polyquaternium-85, polyquaternium-86, polyquaternium-87, polyquaternium-90, polyquaternium-91, polyquaternium-92, or polyquaternium-94; cationic guar gum derivatives such as hydroxypropyl guar hydroxypropyltrimonium chloride or guar hydroxypropyltrimonium chloride; or mixtures thereof.

In some embodiments, the compositions comprise at least one cationic polymer, for example a polyquaternium compound, a cationic guar gum derivative, or a mixture thereof. In certain embodiments, the compositions comprise both a quaternium compound and a cationic guar gum derivative.

In some embodiments, the compositions are free or substantially free of cationic compounds.

When present, the total amount of cationic compounds ranges from about 0.01% to about 10%, such as from about 0.05% to about 5%, from about 0.075% to about 3%, or from about 0.1% to about 1% by weight, relative to the total weight of the composition.

### Silicone(s)

Compositions according to the disclosure may optionally comprise at least one silicone compound. In certain embodiments, non-limiting silicone compounds that can be used include, but are not limited to, polyorganosiloxanes, polyalkylsiloxanes, polyarylsiloxanes, polyalkarylsiloxanes, polyestersiloxanes, and a mixture thereof. For example, dimethicone, cyclomethicone (cyclopentasiloxane), amodimethicone, cetearylamodimethicone, trimethyl silyl amodimethicone, phenyl trimethicone, trimethyl siloxy silicate, polymethylsilsesquioxane, or mixtures thereof may be chosen.

In at least certain embodiments, the compositions are free or essentially free of silicone compounds. For purposes of this disclosure, when the compositions are essentially free of silicone compounds, they comprise less than 0.01% of added silicone compounds by weight, relative to the total weight of the composition.

When present, the total amount of silicones is typically less than about 2%, such as less than about 1.5%, less than about 1% or less than about 0.75%, or less than about 0.5% by weight, relative to the total weight of the composition. For example, the total amount of silicones, when present, may range from about 0.01% to about 2%, such as about 0.025% to about 1.5%, about 0.05% to about 1%, about 0.075% to about 0.75%, or about 0.1% to about 0.5% by weight, relative to the total weight of the composition.

### Additional Nonionic Surfactant(s)

Compositions according to the disclosure may optionally comprise at least one nonionic surfactant in addition to the at least one alkyl(poly)glucoside. However, in some embodiments, the compositions are free or substantially free of nonionic surfactants other than alkyl(poly)glucosides.

By way of example only, if present, the additional nonionic surfactant(s) may be chosen from alkyl and polyalkyl esters of poly(ethylene oxide), alkyl and polyalkyl ethers of poly(ethylene oxide), optionally polyoxyethylenated alkyl and polyalkyl esters of sorbitan, optionally polyoxyethylenated alkyl and polyalkyl ethers of sorbitan, alkyl and polyalkyl esters of sucrose, optionally polyoxyethylenated alkyl and polyalkyl esters of glycerol, and optionally polyoxyethylenated alkyl and polyalkyl ethers of glycerol, or mixtures thereof.

Exemplary and nonlimiting alkyl and polyalkyl esters of poly(ethylene oxide) include those containing at least one C8-C30 alkyl radical, with a number of ethylene oxide (EO) units ranging from 2 to 200. Mention may be made, for example, of PEG-20 stearate, PEG-40 stearate, PEG-100 stearate, PEG-20 laurate, PEG-8 laurate, PEG-40 laurate, PEG-55 propylene glycol oleate, PEG-150 distearate, PEG-7 cocoate, PEG-9 cococate, PEG-8 oleate, PEG-10 oleate, and PEG-40 hydrogenated castor oil.

Exemplary and nonlimiting alkyl and polyalkyl ethers of poly(ethylene oxide) include those containing at least one C8-C30 alkyl radical, with a number of ethylene oxide (EO) units ranging from 3 to 200. Mention may be made, for example, of laureth-3, laureth-4, laureth-5, laureth-7, laureth-23, ceteth-5, ceteth-7, ceteth-15, ceteth-23, oleth-5, oleth-7, oleth-10, oleth-12, oleth-20, oleth-50, phytosterol 30 EO, steareth-6, steareth-20, steareth-21, steareth-40, steareth-100, beheneth 100, ceteareth-7, ceteareth-10, ceteareth-15, ceteareth-25, pareth-3, pareth-23, C12-15 pareth-3, C12-13 pareth-4, C12-13 pareth-23, trideceth-3, trideceth-4, trideceth-5, trideceth-6, trideceth-7 and trideceth-10, and mixtures thereof.

Exemplary and nonlimiting polyoxyethylenated alkyl and polyalkyl esters of sorbitan include those with a number of ethylene oxide (EO) units ranging from 0 to 100. Mention may be made, for example, of sorbitan laurate, sorbitan laurate 4 EO, sorbitan laurate 20 EO (polysorbate 20), sorbitan palmitate 20 EO (polysorbate 40), sorbitan stearate 20 EO (polysorbate 60), sorbitan oleate 20 EO (polysorbate 80) and sorbitan trioleate 20 EO (polysorbate 85).

Exemplary and nonlimiting polyoxyethylenated alkyl and polyalkyl ethers of sorbitan include those with a number of ethylene oxide (EO) units ranging from 0 to 100.

Exemplary and nonlimiting alkyl and polyalkyl esters of sucrose that may be mentioned are Crodesta™ F150, sucrose monolaurate sold under the name Crodesta SL 40, and the products sold by Ryoto Sugar Ester, for instance sucrose palmitate sold under the reference Ryoto™ Sugar Ester P1670, Ryoto™ Sugar Ester LWA 1695 or Ryoto Sugar™ Ester 01570. Sucrose monooleate, monomyristate and monostearate are also suitable for use.

Exemplary and nonlimiting (poly)oxyethylenated alkyl and polyalkyl esters of glycerol include those with a number of ethylene oxide (EO) units ranging from 0 to 100 and a number of glycerol units ranging from 1 to 30. Mention may be made, for example, of hexaglyceryl monolaurate, PEG-55 propylene glycol oleate, PEG-30 glyceryl stearate, polyglyceryl-2 laurate, polyglyceryl-10 laurate, polyglyceryl-10 stearate, polyglyceryl-10 oleate, PEG-7 glyceryl cocoate and PEG-20 glyceryl isostearate.

Exemplary and nonlimiting (poly)oxyethylenated alkyl and polyalkyl ethers of glycerol include those with a number of ethylene oxide (EO) units ranging from 0 to 100 and a number of glycerol units ranging from 1 to 30. Examples that may be mentioned include Nikkol Batyl Alcohol 100 and Nikkol Chimyl Alcohol 100.

### Thickening Agent(s)/Rheology Modifier(s)

The compositions may optionally further comprise at least one thickening agent (also referred to as rheology or viscosity modifying agents). As non-limiting examples, polysaccharide-based thickeners, which are polymers having monosaccharides or disaccharides as base units, may be chosen. The polysaccharide thickeners which can be used in the compositions according to the present invention include, by way of example only, gums, celluloses, starches, or mixtures thereof.

Non-limiting examples of gums include acacia, agar, algin, alginic acid, ammonium alginate, amylopectin, calcium alginate, calcium carrageenan, carnitine, carrageenan, dextrin, gelatin, gellan gum, guar gum, hyaluronic acid, hydrated silica, hydroxypropyl chitosan, hydroxypropyl guar, karaya gum, kelp, locust bean gum, natto gum, potassium alginate, potassium carrageenan, propylene glycol alginate, sclerotium gum, sodium carboxymethyl dextran, sodium carrageenan, tragacanth gum, xanthan gum, and biosacharide gum. Modified gums or derivatives of gums may also be used, such as, for example, deacylated gellan gum, welan gum, or hydroxypropylated guar gum, such as Jaguar HP 105 sold by Rhodia.

Non-limiting examples of celluloses include hydroxyalkylcelluloses, such as hydroxyethylcellulose, hydroxypropylmethylcellulose, or hydropropylcelluloses, which may or may not contain a fatty chain. One particularly suitable hydroxypropylmethylcellulose is Methocel F4M sold by Dow Chemicals (INCI name: hydroxypropylmethylcellulose). Celluloses modified with groups comprising one or more nonionic fatty chains that can be used include hydroxyethylcelluloses, for example nonionic hydroxyethylcelluloses, modified by groups comprising at least one fatty chain, such as alkyl, arylalkyl or alkylaryl groups, or their mixtures, and in which the alkyl groups are preferably C8-C22 alkyl groups, such as the product NATROSOL™ Plus Grade 330 CS (C16 alkyls), sold by Aqualon, corresponding to the INCI name cetylhydroxyethylcellulose, or the product BERMOCOLL® EHM 100 sold by Berol Nobel, and those modified with alkylphenyl polyalkylene glycol ether groups, such as the product AMERCELL POLYMER® HM-1500 (nonylphenyl polyethylene glycol (15) ether) sold by Amerchol that corresponds to the INCI name nonoxynyl hydroxyethylcellulose.

Non-limiting examples of starches include modified starches, starch-based polymers, methylhydroxypropyl starch, potato starch, wheat starch, rice starch, starch crosslinked with octenyl succinic anhydride, starch oxide, dialdehyde starch, dextrin, British gum, acetyl starch, starch phosphate, carboxymethyl starch, hydroxyethyl starch, and hydroxypropyl starch.

In various exemplary embodiments, the total amount of the one or more polysaccharide thickener may vary, but is typically ranges from about 0.01% to about 5%, including all subranges therebetween, such as from about 0.01% to about 4%, from about 0.01% to about 3%, from about 0.01% to about 2%, from about 0.01% to about 1.5%, from about 0.01% to about 1%, from about 0.01% to about 0.5%, from about 0.1% to about 5%, from about 0.1% to about 4%, from about 0.1% to about 3%, from about 0.1% to about 2%, from about 0.1% to about 1.5%, from about 0.1% to about 1%, or from about 0.1% to about 0.5%, by weight, relative to the total weight of the composition. In at least certain embodiments, the composition comprises a thickener in an amount less than about 1%.

### Solvent(s)

Compositions according to the disclosure comprise at least one solvent. The solvent may be chosen from water, non-aqueous solvents, or mixtures thereof.

In some embodiments, the solvent comprises, consists essentially of, or consists of water. The total amount of water in the compositions may vary depending on the type of composition and the desired consistency, viscosity, etc. In various embodiments, water is present in the compositions in an amount ranging from about 60% to about 95%, such as about 65% to about 90%, about 70% to about 85%, or about 75% to about 85% by weight, relative to the total weight of the composition.

In certain embodiments, the composition comprises one or more non-aqueous solvents, for example, glycerin, C₁₋₄ alcohols, organic solvents, fatty alcohols, fatty ethers, fatty esters, polyols, glycols, vegetable oils, mineral oils, liposomes, laminar lipid materials, or any a mixture thereof. Non-limiting examples of solvents which may be used include alkanediols such as glycerin, 1,2,6-hexanetriol, trimethylolpropane, ethylene glycol, propylene glycol, diethylene glycol, triethylene glycol, tetraethylene glycol, pentaethylene glycol, dipropylene glycol, 2-butene-1,4-diol, 2-ethyl-1,3-hexanediol, 2-methyl-2,4-pentanediol, caprylyl glycol, 1,2-hexanediol, 1,2-pentanediol, and 4-methyl-1,2-pentanediol; alkyl alcohols having 1 to 4 carbon atoms such as ethanol, methanol, butanol, propanol, and isopropanol; glycol ethers such as ethylene glycol monomethyl ether, ethylene glycol monoethyl ether, ethylene glycol monobutyl ether, ethylene glycol monomethyl ether acetate, diethylene glycol monomethyl ether, diethylene glycol monoethyl ether, diethylene glycol mono-n-propyl ether, ethylene glycol mono-iso-propyl ether, diethylene glycol mono-iso-propyl ether, ethylene glycol mono-n-butyl ether, ethylene glycol mono-t-butyl ether, diethylene glycol mono-t-butyl ether, 1-methyl-1-methoxybutanol, propylene glycol monomethyl ether, propylene glycol monoethyl ether, propylene glycol mono-t-butyl ether, propylene glycol mono-n-propyl ether, propylene glycol mono-iso-propyl ether, dipropylene glycol monomethyl ether, dipropylene glycol monoethyl ether, dipropylene glycol mono-n-propyl ether, and dipropylene glycol mono-iso-propyl ether; 2-pyrrolidone, N-methyl-2-pyrrolidone, 1,3-dimethyl-2-imidazolidinone, formamide, acetamide, dimethyl sulfoxide, sorbit, sorbitan, acetine, diacetine, triacetine, sulfolane, and a mixture thereof.

The total amount of solvent may range from about 60% to about 98% by weight, relative to the total weight of the composition, including all ranges and subranges therebetween. For example, in one embodiment, the total amount of solvent may be about 70% to about 95%, about 70% to about 90%, about 75% to about 85%, or about 75% to about 80% by weight, relative to the total weight of the composition.

### Auxiliary Components

Compositions according to the disclosure may optionally comprise any auxiliary component suitable for use in such compositions. Such components may include, but are not limited to, dyes/pigments, humectants and moisturizing agents, fatty substances, fillers, structuring agents, shine agents, antioxidants or reducing agents, penetrants, sequestrants, fragrances, buffers, dispersants, plant extracts, preserving agents, opacifiers, sunscreen agents, vitamins, pH adjusting agents, and antistatic agents.

Optional auxiliary components may be present in the composition in a total amount ranging up to about 15%, such as up to about 10%, up to about 5%, up to about 3%, up to about 2%, or up to about 1% by weight, relative to the total weight of the composition. For example, compositions according to the disclosure may comprise a total amount of auxiliary components ranging from about 0.001% to about 5%, from about 0.005% to about 4%, from about 0.01% to about 3%, from about 0.05% to about 2.5%, or from about 0.1% to about 2% by weight, relative to the total weight of the composition.

The compositions may be transparent, semi-transparent, or opaque, and their viscosities may vary but may be similar to or greater than that of conventional cleansing, shampooing, and/or conditioning compositions. For example, in some embodiments, the compositions according to the present disclosure may range from thickened liquid to a thick gel-like texture. Accordingly, in some instances, the viscosity of a composition disclosed herein may be from about 800 cPs to about 20,000 cPs, such as from about 800 cPs to about 15,000 cPs, 800 cPs to about 12,000 cPs, from about 1000 cPs to about 15,000 cPs, from about 1000 cPs to about 12,000 cPs, from about 1500 cPs to about 15,000 cPs, from about 1500 cPs to about 12,000 cPs, from about 2000 cPs to about 15,000 cPs, or from about 2000 cPs to about 12,000 cPs, when measured at 25° C. using a Brookfield Viscometer DV-II+ Pro, RV spindle at 10 rpm.

The compositions may have a pH less than or equal to 9, such from about 3 to about 8, from about 4 to about 7, from about 5 to about 6.5, or from about 5 to about 6.

In various embodiments, the compositions according to the present disclosure are stable, meaning that no phase separation or significant change in pH or viscosity is seen when stored at a temperature ranging from about 4° C. to about 45° C., such as from about 10° C. to about 37° C., or from about 20° C. to about 30° C., for at least about 8 weeks.

In at least some embodiments, compositions according to the disclosure may be mild, display good foaming properties, good detangling and combing properties, good antistatic properties, and/or good stability. The compositions may impart one or more properties such as smoothness, conditioning, excellent detangling, anti-frizz, ease of shaping and/or combing, anti-static, clean and/or smooth appearance, with no weigh-down.

II. Methods

The present disclosure also relates to methods for cleansing and/or conditioning keratin materials, especially the hair, skin, and/or the scalp, with the compositions disclosed herein. Without limitation, methods of cleansing and/or conditioning keratin materials according to the disclosure may include applying a sufficient amount, or an effective amount, of a composition disclosed herein to a keratin material, such as hair, skin, or scalp, which may be wet, damp, or dry, optionally allowing the composition to remain on the keratin material for a desired amount of time, and optionally rinsing the composition from the keratin material. The composition may optionally be lathered before application to the keratin materials, e.g. in the hands, or may be lathered while on the keratin materials.

Due to the cleansing and conditioning properties of the compositions, in some instances, the compositions may be designated as a “shampoo,” a “conditioning shampoo,” or an “all-in-one conditioning and shampooing composition.” The compositions may also be both a hair and face and/or body wash, or a face and/or body wash. In certain embodiments, compositions of the instant disclosure are particularly useful for cleansing and conditioning hair. Additionally, the compositions provide a variety of desirable cosmetic and styling benefits to the hair, for example, smoothness without weight-down, detangling, and anti-frizz. As such, the compositions are useful in methods for cleansing hair, methods of conditioning hair, and methods for imparting smoothness, detangling, and/or frizz control to hair. Accordingly, the instant disclosure encompasses methods for treating hair with the compositions of the instant disclosure.

Such methods typically include applying an effective amount of a composition of the instant disclosure to the hair, allowing the composition to remain on the hair for a period of time, and subsequently rinsing the composition from the hair, followed by allowing the hair to air dry or drying the hair with a hair dryer which blows air through the hair and accelerates drying. Usually, the composition is merely allowed to remain on the hair for a period of time sufficient to incorporate the composition throughout the hair, for example, by lathering the composition throughout the hair using one's hands.

The amount of time is sufficient for the composition to interact with the hair and any dirt, oil, contamination, etc., that may exist on the hair so that when rinsed, the dirt, oil, contamination, etc., can be effectively removed from the hair and the conditioning agents of the composition can interact with the hair to condition it. Thus, the composition may be allowed to remain on the hair for about 5 seconds to about 30 minutes, about 5 seconds to about 15 minutes, about 5 seconds to about 10 minutes, about 5 seconds to about 5 minutes, about 10 seconds to about 5 minutes, or about 10 seconds to about 3 minutes. The composition is then rinsed from the hair, and the hair allowed to dry.

As is common when using shampoo and/or conditioning compositions, the hair may be wetted or rinsed with water prior to application of a composition disclosed herein. Having water already in the hair may be helpful for creating lather when applying the compositions because the water interacts with the surfactants.

Having described the many embodiments of the present invention in detail, it will be apparent that modifications and variations are possible without departing from the scope of the disclosure defined in the appended claims. Furthermore, it should be appreciated that all examples in the present disclosure, while illustrating many embodiments of the disclosure, are provided as non-limiting examples and are, therefore, not to be taken as limiting the various aspects so illustrated. It is to be understood that all definitions herein are provided for the present disclosure only.

Unless otherwise expressly stated, it is in no way intended that any method set forth herein be construed as requiring that its steps be performed in a specific order. Accordingly, where a method claim does not expressly recite an order to be followed by its steps or it is not specifically stated in the claims or descriptions that the steps are to be limited to a specific order, it is no way intended that any particular order be inferred.

Other than in the operating examples, or where otherwise indicated, all numbers expressing quantities of ingredients and/or reaction conditions are to be understood as being modified in all instances by the term “about,” meaning within 10% of the indicated number (e.g. “about 10%” means 9%-11%, “about 2%” means 1.8%-2.2%, and so on).

It should be understood that the precise numerical values used in the specification, including the examples and claims, form additional embodiments of the invention, and are intended to include any ranges which can be narrowed to any to end points disclosed within the exemplary ranges and values provided. Efforts have been made to ensure the accuracy of the numerical values disclosed. However, any measured value can inherently contain certain errors resulting from the standard deviation found in its respective measuring technique.

As used herein, the terms “comprising,” “having,” and “including” (or “comprise,” “have,” and “include”) are used in their open, non-limiting sense.

As used herein, the use of the singular includes the plural unless specifically stated otherwise. The singular forms “a,” “an,” “the,” and “at least one” are understood to encompass the plural as well as the singular unless the context clearly dictates otherwise. The expression “one or more” and “at least one” are interchangeable and expressly include individual components as well as mixtures/combinations. Likewise, the term “a salt thereof” also relates to “salts thereof.” Thus, where the disclosure refers to “at least one element selected from the group consisting of A, B, C, D, E, F, a salt thereof, or mixtures thereof,” it indicates that that one or more of A, B, C, D, and F may be included, one or more of a salt of A, a salt of B, a salt of C, a salt of D, a salt of E, and a salt of F may be included, or a mixture of any two or more of A, B, C, D, E, F, one or more salts of A, one or more salts of B, one or more salts of C, one or more salts of D, one or more salts of E, and one or more salts of F may be included.

The term “and/or” should be understood to include both the conjunctive and the disjunctive. For example, “water and/or non-aqueous solvents” means “water and non-aqueous solvents” as well as “water or non-aqueous solvents,” and expressly covers instances of either.

As used herein, the phrases “and mixtures thereof,” “and a mixture thereof,” “and combinations thereof,” “and a combination thereof,” “or mixtures thereof,” “or a mixture thereof,” “or combinations thereof,” and “or a combination thereof,” are used interchangeably to denote that the listing of components immediately preceding the phrase, such as “A, B, C, D, or mixtures thereof” signify that the component(s) may be chosen from A, from B, from C, from D, from A+B, from A+B+C, from A+D, from A+C+D, etc., without limitation on the variations thereof. Thus, the components may be used individually or in any combination thereof.

For purposes of the disclosure, it should be noted that to provide a more concise description, some of the quantitative expressions given herein are not qualified with the term “about.” It is understood that whether the term “about” is used explicitly or not, every quantity given herein is meant to refer to the actual given value, and it is also meant to refer to the approximation to such given value that would reasonably be inferred based on the ordinary skill in the art, including approximations due to the experimental and/or measurement conditions for such given value.

All ranges and amounts given herein are intended to include sub-ranges and amounts using any disclosed point as an end point, and all endpoints are intended to be included unless expressly stated otherwise. Thus, a range of “1% to 10%, such as 2% to 8%, such as 3% to 5%,” is intended to encompass ranges of “1% to 8%,” “1% to 5%,” “2% to 10%,” and so on. All numbers, amounts, ranges, etc., are intended to be modified by the term “about,” whether or not expressly stated, unless expressly stated otherwise. Similarly, a range given of “about 1% to 10%” is intended to have the term “about” modifying both the 1% and the 10% endpoints. The term “about” is used herein to indicate a difference of up to +/−10% from the stated number, such as +/−9%, +/−8%, +/−7%, +/−6%, +/−5%, +/−4%, +/−3%, +/−2%, or +/−1%. Likewise, all endpoints of ranges are understood to be individually disclosed, such that, for example, a range of 1:2 to 2:1 is understood to disclose a ratio of both 1:2 and 2:1.

Throughout the disclosure, if the terms “mixtures thereof,” “a mixture thereof,” “a combination thereof,” or variants thereof are used following a list of elements, as shown in the following example where letters A-F represent the elements: “one or more elements selected from the group consisting of A, B, C, D, E, F, or mixtures thereof,” it does not require that the mixture include all of A, B, C, D, E, and F (although all of A, B, C, D, E, and F may be included). Rather, it indicates that a mixture of any two or more of A, B, C, D, E, and F can be included. In other words, it is equivalent to the phrase “one or more elements selected from A, B, C, D, E, F, and a mixture of any two or more of A, B, C, D, E, and F.”

“At least one,” as used herein, means one or more and thus includes individual components as well as mixtures/combinations.

“Active material” or “weight” as used herein with respect to the percent amount of an ingredient or raw material, refers to 100% activity of the ingredient or raw material.

All amounts and ratios herein are given by weight, based upon the total weight of the composition, unless otherwise indicated. Unless otherwise indicated, all percentages herein are by weight of active material.

As used herein, the term “salts” referred to throughout the disclosure may include salts having a counterion such as an alkali metal, alkaline earth metal, or ammonium counterion. This list of counterions, however, is non-limiting. Salts also include a dissociated form of a compound, e.g. in an aqueous solution.

“Antioxidant” refers to a chemical compound, an enzyme or other organic molecule which prevents free radicals from causing oxidation of molecules such as are found in keratinous materials. The antioxidant, by reacting with the oxidant, protects such molecules from being damaged. Examples of antioxidants include without limitation, polyphenols, vitamins A, C, E, carotenoids, and certain minerals. In some specific examples, antioxidants include ascorbic acid, dihydrochalone, zinc PCA, baicalin, ferulic acid, pine bark extract, polydatin and ellagic acid.

As used herein, the terms “applying a composition onto keratin substrates,” “applying a composition onto hair,” and variations of these phrases are intended to mean contacting the substrates or hair with at least one of the compositions according to the disclosure, in any manner.

“Cosmetically acceptable” means compatible with any keratinous tissue. For example, “cosmetically acceptable carrier” means a carrier that is compatible with any keratinous tissue.

“Keratinous material,” as used herein, includes, but is not limited to, skin, hair, and nails, and also includes “keratinous substrate,” “keratinous tissue,” and “keratinous fibers,” which may be human keratinous material, and may be chosen from, for example, hair, such as hair on the human head, or hair comprising of eyelashes or hair on the body.

As used herein, the term “conditioning” means imparting to keratin materials at least one property chosen from combability, moisture-retentivity, luster, shine, and softness. The state of conditioning can be evaluated by any means known in the art, such as, for example, measuring, and comparing, the ease of combability of the treated hair and of the untreated hair in terms of combing work, and consumer perception.

As used herein, “cosmetic composition” encompasses many types of compositions for application to keratin materials such as skin or hair, for example, hair lotions, hair creams, hair gel creams, hair conditioners, hair masques (masks), etc., which can be used either as leave-on or rinse-off treatments or products.

As used herein, the term “organic” means a material that is produced substantially without or essentially without the use of synthetic materials. The term “substantially without” or “essentially without” as used herein means the specific material may be used in a manufacturing process in small amounts that do not materially affect the basic and novel characteristics of the compositions according to the disclosure. The term “substantially without” or “essentially without” as used herein may also mean that the specific material is not used in a manufacturing process but may still be present in a raw material that is included in the composition.

As used herein, the term “salts” refers to throughout the disclosure may include salts having a counter-ion such as an alkali metal, alkaline earth metal, or ammonium counterion. This list of counterions, however, is non-limiting.

Unless specifically indicated otherwise, as used herein, the terms “substantially free” or “essentially free,” which are used interchangeably, mean that the specific material may be present in small amounts that do not materially affect the basic and novel characteristics of the compositions according to the disclosure. For instance, there may be less than 2% by weight of a specific material added to a composition, based on the total weight of the compositions (provided that an amount of less than 2% by weight does not materially affect the basic and novel characteristics of the compositions according to the disclosure. Similarly, the compositions may include less than 2%, less than 1.5%, less than 1%, less than 0.5%, less than 0.1%, less than 0.05%, or less than 0.01%, or none of the specified material. Furthermore, all components that are positively set forth in the instant disclosure may be negatively excluded from the claims, e.g., a claimed composition may be “free,” “essentially free” (or “substantially free”) of one or more components that are positively set forth in the instant disclosure. The term “substantially free” or “essentially free” as used herein may also mean that the specific material is not added to the composition but may still be present in a raw material that is included in the composition, in an amount that that does not materially affect the basic and novel characteristics of the compositions according to the disclosure.

As used herein, the term “sulfate-based surfactant” also means “sulfate-containing surfactant.” Thus, the term “essentially free of sulfate-based surfactant” also means “essentially free of sulfate-containing surfactant.”

As used herein, the term “surfactants,” as well as any specifically-identified surfactants, includes salts of the surfactants even if not explicitly stated.

As used herein, the term “surfactant system” refers to a combination of different surfactants. For example, the term “anionic surfactant system” refers to a combination of more two or more different anionic surfactants.

As used herein, the term “synthetic” means a material that is not of natural origin. The term “natural” and “naturally-sourced” means a material of natural origin, such as derived from plants, which also cannot be subsequently chemically or physically modified. “Plant-based” means that the material came from a plant.

As used herein, the term “treat” (and its grammatical variations) refers to the application of the compositions of the present disclosure onto the surface of keratin materials, such as hair.

“Substituted,” as used herein, means comprising at least one substituent. Non-limiting examples of substituents include atoms, such as oxygen atoms and nitrogen atoms, as well as functional groups, such as hydroxyl groups, ether groups, alkoxy groups, acyloxyalkyl groups, oxyalkylene groups, polyoxyalkylene groups, carboxylic acid groups, amine groups, acylamino groups, amide groups, halogen containing groups, ester groups, thiol groups, sulphonate groups, thiosulphate groups, siloxane groups, and polysiloxane groups. The substituent(s) may be further substituted.

Unless otherwise expressly stated, it is in no way intended that any method set forth herein be construed as requiring that its steps be performed in a specific order. Accordingly, where a method does not expressly recite that a particular order of steps must be followed or it is not otherwise specifically stated that the steps are to be limited to a specific order, it is no way intended that any particular order be inferred.

It should also be understood that the precise numerical values used in the specification and claims form additional embodiments of the disclosure, and are intended to include any ranges which may be narrowed to any two end points disclosed within the exemplary ranges and values provided, as well as the specific end points themselves.

Notwithstanding that the numerical ranges and parameters setting forth the broad scope of the disclosure are approximations, unless otherwise indicated the numerical values set forth in the specific examples are reported as precisely as possible. Any numerical value, however, inherently contains certain errors necessarily resulting from the standard deviation found in their respective testing measurements. The examples that follow serve to illustrate embodiments of the disclosure without, however, being limiting in nature.

### EXAMPLES

The following examples are intended to be non-limiting and explanatory in nature only. In the Examples, amounts are expressed in percentage by weight (wt %) of active materials, relative to the total weight of the composition.

### Example 1—Sulfate-Free Cleansing Compositions

The following inventive compositions were prepared according to the formulations set forth in Table 1 below.

TABLE 1
Cleansing Compositions

| | Inventive Compositions | | |
|---|---|---|---|
| | 1A | 1B | 1C |
| SODIUM CHLORIDE | 0.99 | 0.99 | 0.96 |
| GLYCOL DISTEARATE | 1.20 | 1.20 | 0.60 |
| POLYQUATERNIUM-7 | 0.36 | 0.36 | 0.36 |
| HYDROXYETHEYLCELLULOSE | | 0.10 | |
| AMODIMETHICONE | 0.2 | 0.2 | 0.4 |
| COCO-BETAINE | 0.24 | 0.24 | 0.12 |
| LAURETH-5 CARBOXYLIC ACID | 0.90 | 0.90 | 0.90 |
| COCAMIDOPROPYL BETAINE | 5.1 | 5.1 | 5.1 |
| SODIUM COCOYL ISETHIONATE | 6.0 | 6.0 | 6.0 |
| SODIUM METHYL COCOYL | 1.55 | 1.55 | 1.55 |
| TAURATE | | | |
| DECYL GLUCOSIDE | 2.12 | 2.12 | 2.12 |
| ADDITIVES (fragrance, preservatives, | <3 | <3 | <3 |
| pH and viscosity adjusters) | | | |
| SOLVENT (water + non-aqueous | QS | QS | QS |
| solvents) | | | |

Inventive compositions 1A-1C were in liquid form, were semi-transparent or opaque, and were stable. Each of inventive compositions 1A-1C had a pH in the range of 5-6.5, and viscosities in the range of about 1600-3400 centipoise (cPs) when measured at 25° C. using a Brookfield Viscometer DV-II+ Pro, RV spindle at 10 rpm.

Each of inventive compositions 1A-1C demonstrated ease/quickness in generating foam, good foam quality (creamy, lush, and stable), and abundance.

### Example 2—Comparison with Comparative Sulfate-Containing Shampoo

To evaluate performance differences between sulfate-free cleansing shampoos according to the disclosure and sulfate-containing shampoos, inventive composition 1B from Table 1 above was tested on hair on the heads of human volunteers against a commercially-available sulfate-containing shampoo (C1) having the ingredients listed in Table 2.

TABLE 2
Commercial Sulfate-Containing Shampoo
Commercial Product: C1

| | |
|---|---|
| Ingredients | Aqua/Water/Eau, Sodium Laureth Sulfate, Cocamidopropyl Betaine, |
| as listed on | Dimethicone, Sodium Chloride, Parfum/Fragrance, Hexylene Glycol, |
| package | Citric Acid, Sodium Benzoate, Sodium Hydroxide, Amodimethicone, |
| | Carbomer, Guar Hydroxypropyltrimonium Chloride, Trideceth-10, |
| | Glycerin, Salicylic Acid, Glycol Distearate, Ricinus Communis Seed |
| | Oil/Castor Seed Oil, Mica, PEG-100 Stearate, Niacinamide, Panthenol, |
| | Phenoxyethanol, Steareth-6, Hexyl Cinnamal, Benzyl Salicylate, Linalool, |
| | Benzyl Alcohol, Coco-Betaine, Trideceth-3, Limonene, CI |
| | 77891/Titanium Dioxide, Alpha-lsomethyl Ionone, Coumarin, Citronellol, |
| | Hydrolyzed Wheat Protein, Hydroxycitronellal, Geraniol, Hydrolyzed Corn |
| | Protein, Hydrolyzed Soy Protein, Acetic Acid, Fumaric Acid, |
| | Hydroxypropyltrimonium Hydrolyzed Wheat Protein. |

Equal amounts of the inventive and comparative cleansing compositions were applied to opposite halves of a head of damp hair with no prior product and worked into a lather with water, the hair was rinsed, and then blown dry.

As evaluated, the performance of inventive composition 1B provided greater smoothness of foam during application, improved smoothness on wet hair during and after rinsing, greater ease in blow drying, and more discipline, body, smoothness, volume, and sealed ends when dry, compared to composition C1. Additionally, inventive composition 1B surprisingly demonstrated ease/quickness in generating foam, good foam quality (creamy, lush, and stable), and abundance of foam comparable to that of composition C1, despite the absence of sulfates.

Based on the above Examples, it was demonstrated that compositions according to the disclosure surprisingly provide a sulfate-free shampoo with excellent foaming and performance properties.

1-29. (canceled)

30. A composition for treating keratin materials comprising:
(a) at least one amphoteric surfactant;
(b) an anionic surfactant system comprising:
i. at least one acyl taurate;
ii. at least one acyl isethionate; and
iii. at least one additional anionic surfactant chosen from alkoxylated monoacids, alkyl sulfosuccinates, alkyl ether sulfosuccinates, or mixtures thereof;
(c) at least one nonionic surfactant chosen from alkyl(poly)glycosides;
(d) optionally, at least one cationic compound; and
(e) sodium chloride;
wherein the weight ratio of the (b) anionic surfactant system to (e) sodium chloride ranges from about 5:1 to about 15:1; and
wherein the composition is essentially free of sulfate-based surfactants.

31. The composition according to claim 30, wherein the weight ratio of the (b) anionic surfactant system to (e) sodium chloride ranges from about 8:1 to about 12:1.

32. The composition according to claim 30, further comprising (f) at least one silicone compound, wherein the total amount of silicone compounds is less than about 2% by weight, relative to the total weight of the composition.

33. The composition of claim 30, wherein the total amount of cationic compounds ranges from about 0.01% to about 10% by weight, relative to the total weight of the composition.

34. The composition of claim 30, comprising at least one amphoteric surfactant chosen from compounds of formulae (I), (II), (Ill), or (IV): 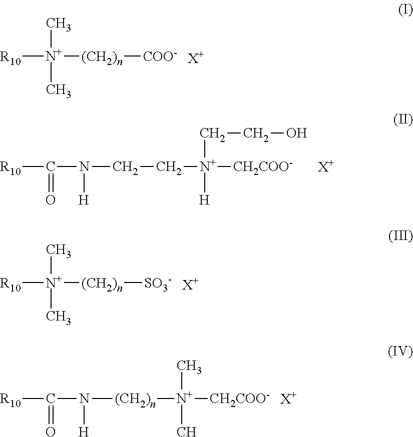
wherein:
R₁₀ is an alkyl group having from 8 to 18 carbon atoms;
n is an integer ranging from 1 to 3; and
X⁺ is a cationic counterion.

35. The composition of claim 30, comprising at least one amphoteric surfactant chosen from (C₈-C₂₀) alkylbetaines, sulfobetaines, (C₈-C₂₀) alkylamido (C₆-C₈) alkylbetaines, (C₈-C₂₀) alkylamido (C₆-C₈) alkylsulfobetaines, salts thereof, or mixtures thereof.

36. The composition of claim 30, comprising at least one amphoteric surfactant chosen from coco betaine, cocoamidopropyl betaine, lauryl betaine, laurylhydroxy sulfobetaine, lauryldimethyl betaine, behenyl betaine, capryl/capramidopropyl betaine, stearyl betaine, salts thereof, or mixtures thereof.

37. The composition of claim 30, comprising at least one acyl taurate chosen from compounds of formula (V): 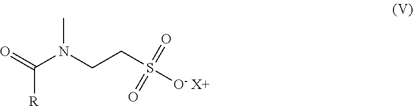
wherein:
R is chosen from hydrogen or a saturated or unsaturated, linear or branched alkyl chain having from 1 to 24 carbon atoms; and
X⁺ is a cationic counterion.

38. The composition of claim 30, comprising at least one acyl taurate chosen from sodium methyl lauroyl taurate, sodium methyl cocoyl taurate, or mixtures thereof.

39. The composition of claim 30, comprising at least one alkoxylated monoacid chosen from compounds of formula (VI):
R—O[CH₂O]ᵤ[(CH₂)ₓCH(R′)(CH₂)_{y}(CH₂)_{z}O]ᵥ[CH₂CH₂O]_{w}CH₂—COOH (VI)
wherein:
R is a hydrocarbon radical containing from 6 to 40 carbon atoms;
R′ represents hydrogen or alkyl;
u, v, and w, independently of one another, represent numbers ranging from 0 to 60;
x, y, and z, independently of one another, represent numbers ranging from 0 to 13; and
the sum of x+y+z>0.

40. The composition of claim 30, comprising at least one alkoxylated monoacid chosen from ceteareth-2 carboxylic acid, ceteareth-10 carboxylic acid, coceth-7 carboxylic acid, laureth-4 carboxylic acid, laureth-5 carboxylic acid, laureth-6 carboxylic acid, myreth-2 carboxylic acid, myreth-3 carboxylic acid, myreth-4 carboxylic acid, myreth-5 carboxylic acid, myreth-6 carboxylic acid, steareth-2 carboxylic acid, steareth-4 carboxylic acid, steareth-5 carboxylic acid, steareth-6 carboxylic acid, oleth-2 carboxylic acid, oleth-4 carboxylic acid, salts thereof, or mixtures thereof.

41. The composition of claim 30, comprising at least one acyl isethionate chosen from compounds of formula (VII): 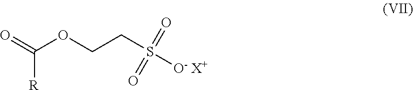
wherein:
R is chosen from hydrogen or a saturated or unsaturated, linear or branched alkyl chain having from 1 to 24 carbon atoms; and
X⁺ is a cationic counterion.

42. The composition of claim 30, comprising at least one acyl isethionate chosen from sodium cocoyl isethionate, sodium cocoyl methyl isethionate, sodium lauroyl isethionate, sodium lauroyl methyl isethionate, sodium oleoyl isethionate, sodium oleoyl methyl isethionate, sodium stearoyl isethionate, sodium stearoyl methyl isethionate, sodium myristoyl isethionate, sodium myristoyl methyl isethionate, sodium palmitoyl isethionate, sodium palmitoyl methyl isethionate, a blend of stearic acid and sodium cocoyl isethionate, ammonium cocoyl isethionate, ammonium cocoyl methyl isethionate, or mixtures thereof.

43. The composition of claim 30, comprising at least one alkyl(poly)glycoside chosen from compounds of formula (VIII):
R₁O—(R₂O)ₜ-(G)ᵥ (VIII)
wherein:
R₁ represents a linear or branched, saturated or unsaturated alkyl group, containing from about 8 to 24 carbon atoms, or an alkyl phenyl group in which the linear or branched alkyl radical contains from 8 to 24 carbon atoms;
R₂ represents an alkylene group containing from about 2 to 4 carbon atoms;
G represents a saccharide unit containing 5 or 6 carbon atoms;
t is a number ranging from 0 to 10; and
v is a number ranging from 1 to 15.

44. The composition of claim 30, comprising at least one alkyl(poly)glycoside chosen from decylglucoside, laurylglucoside, cocoglucoside, caprylylglucoside, caprylyl/capryl glucoside, or mixtures thereof.

45. The composition of claim 30, wherein the composition is free of silicone compounds.

46. A composition for treating keratin materials comprising:
(a) at least one amphoteric surfactant, wherein the total amount of amphoteric surfactants ranges from about 1% to about 15%;
(b) an anionic surfactant system comprising:
i. at least one acyl taurate, wherein the total amount of acyl taurates ranges from about 0.1% to about 5%;
ii. at least one acyl isethionate, wherein the total amount of acyl isethionates ranges from about 1% to about 10%; and
iii. at least one additional anionic surfactant chosen from alkoxylated monoacids, wherein the total amount of additional anionic surfactants ranges from about 0.1% to about 5%;
(c) at least one nonionic surfactant chosen from alkyl(poly)glycosides;
(d) at least one cationic polymer; and
(e) sodium chloride,
wherein the weight ratio of the (b) anionic surfactant system to (e) sodium chloride ranges from about 6:1 to about 14:1;
wherein the composition is essentially free of sulfate-based surfactants; and
wherein all amounts are by weight, relative to the total weight of the composition.

47. The composition of claim 45, comprising at least one amphoteric surfactant chosen from (C8-C20) alkylbetaines, sulfobetaines, (C8-C20) alkylamido (C6-C8) alkylbetaines, (C8-C20) alkylamido (C6-C8) alkylsulfobetaines, salts thereof, or mixtures thereof.

48. The composition of claim 45, wherein the composition is essentially free of silicone compounds.

49. A method for cleansing keratin materials comprising:
applying to the keratin materials a composition comprising:
(a) at least one amphoteric surfactant;
(b) an anionic surfactant system comprising:
i. at least one acyl taurate;
ii. at least one acyl isethionate; and
iii. at least one additional anionic surfactant chosen from alkoxylated monoacids, alkyl sulfosuccinates, alkyl ether sulfosuccinates, or mixtures thereof;
(c) at least one nonionic surfactant chosen from alkyl(poly)glycosides;
(d) optionally, at least one cationic compound; and
(e) sodium chloride;
wherein the weight ratio of the (b) anionic surfactant system to (e) sodium chloride ranges from about 5:1 to about 15:1; and
wherein the composition is essentially free of sulfate-based surfactants; and
subsequently rinsing the keratin materials.
